(19) United States
(12) Patent Application Publication
(10) Pub. No.: US 2021/0002678 A1
(43) Pub. Date: Jan. 7, 2021

(54) ENZYMES, CELLS AND METHODS FOR PRODUCTION OF 3-(4-FARNESYLOXYPHENYL)PROPIONIC ACID AND DERIVATIVES THEREOF

(71) Applicant: Manus Bio, Inc., Cambridge, MA (US)

(72) Inventors: Ryan Nicholas PHILIPPE, Cambridge, MA (US); Ajikumar Parayil KUMARAN, Cambridge, MA (US); Christine Nicole S. SANTOS, Cambridge, MA (US)

(21) Appl. No.: 16/982,916

(22) PCT Filed: Mar. 20, 2019
(86) PCT No.: PCT/US2019/023123

§ 371 Date: Sep. 21, 2020

Related U.S. Application Data
(60) Provisional application No. 62/656,678, filed on Apr. 12, 2018.
(60) Provisional application No. 62/645,443, filed on Mar. 20, 2018.

(51) Int. Cl. C12P 7/52; C12N 15/74; A61K 8/97; A61Q 19/02

(52) U.S. Cl. CPC C12P 7/52; A61Q 19/02; A61K 8/97; C12N 15/74

(57) ABSTRACT

The present disclosure provides microbial cells and methods of producing FOPPA resulting from unique biosynthetic pathways, including biosynthetic pathways based on the phenylalanine/tyrosine biosynthetic branch and biosynthetic pathways based on bacteria metabolism. In particular, the present invention provides methods of producing FOPPA in microbial cells. These methods provide a low-cost, sustainable, and environmentally friendly source for FOPPA.

### PRIORITY

This application claims the benefit of, and priority to, U.S. Application No. 62/645,443 filed Mar. 20, 2018 and 62/656,678 filed Apr. 12, 2018, each of which is hereby incorporated by reference in its entirety.

### BACKGROUND

Aronychia derived compounds, including 3-(4-farnesyloxyphenyl)propionic acid (FOPPA) have been described for use as antioxidants, antibacterials, anthelmintics, anti-inflammatories, cancer chemopreventatives, food additives, and/or fragrance components. See US 2011/0318439, which is hereby incorporated by reference in its entirety. Additionally. U.S. Pat. No. 4,939,171, which is hereby incorporated by reference in its entirety, discloses the use of compounds, such as FOPPA, to provide antiseborrhoeic properties. U.S. Pat. No. 9,814,659, which is hereby incorporated by reference in its entirety, discloses that FOPPA can be used to provide skin lightening and photo-protective effects when used on skin.

Given the many potential uses of FOPPA, the compound needs to be produced quickly and efficiently. Moreover, there is a growing need to provide sustainable, and environmental-friendly methods of manufacturing compounds, such as FOPPA.

### BRIEF DESCRIPTION OF THE DRAWINGS

FIG. 1 depicts the chemical structure of 3-(4-farnesyloxyphenyl)propionic acid (FOPPA). FIG. 1 shows the two primary components of FOPPA, a compound derived from farnesyl-PP or farnesol, and phloretate (3-(4-hydroxyphenyl)propionic acid.

FIG. 2 shows summarizes biosynthetic routes to obtain the phloretate precursor of FOPPA. The first approach reconstructs and improves upon plant pathways, including a 7-step pathway from L-phenylalanine or a 6-step pathway from L-tyrosine. The second approach involves the creation of a shortcut pathway using bacterial enzymes, which includes a 3-step pathway from L-tyrosine.

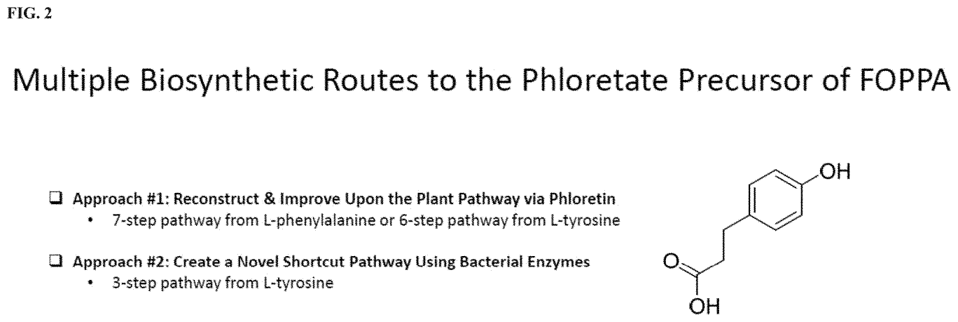

FIG. 3 depicts a biosynthetic pathway to phloretate and FOPPA via phloretin.

FIG. 4 depicts a bacteria-based biosynthetic pathway to phloretate and FOPPA

### DESCRIPTION OF THE INVENTION

The present invention provides methods of producing and making 3-(4-farnesyloxyphenyl)propionic acid (FOPPA), which is also known as 3-(4-farnesyloxyphenyl)propanoic acid, 3-(p-farnesyloxyphenyl)propionic acid, and 3-(p-farnesyloxyphenyl)propanoic acid. FOPPA was originally found in fruit from Acronychia spp. and, specifically, Acronychia acidula (lemon aspen). FOPPA has many beneficial characteristics and can be used in a variety of medical, cosmetic, and food related applications. For example, FOPPA has utility as an agent for skin lightening and photo-protective effects.

The present invention provides methods of producing FOPPA resulting from unique biosynthetic pathways, including biosynthetic pathways based on the phenylalanine/tyrosine biosynthetic branch and biosynthetic pathways based on bacteria metabolism. In particular, the present invention provides methods of producing FOPPA in microbial cells. These methods provide a low-cost, sustainable, and environmentally friendly source for FOPPA.

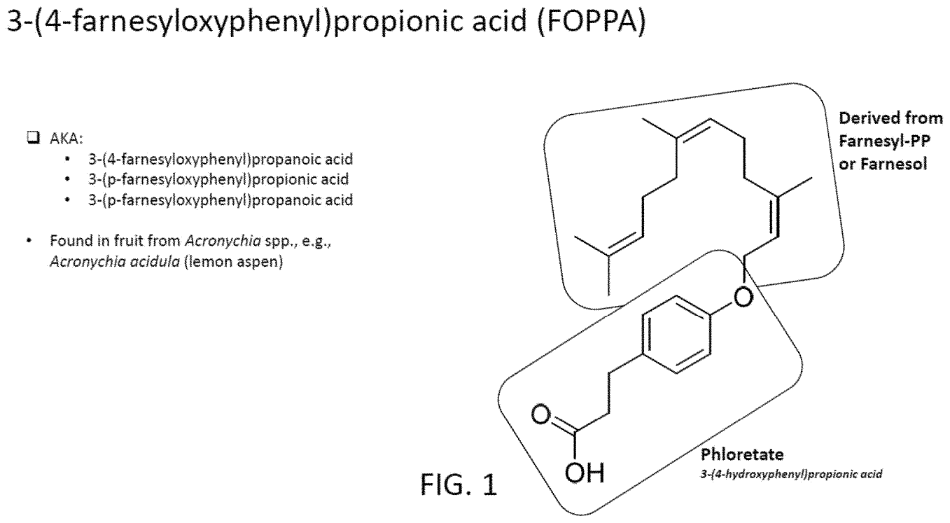

In some aspects, the present invention provides a microbial cell producing 3-(4-farnesyloxyphenyl)propionic acid (FOPPA), or a derivative thereof. The microbial cell comprises an enzyme pathway for the synthesis of a first substrate that is selected from farnesyl pyrophosphate, farnesyl-phosphate, or farnesol; and an enzyme pathway for the synthesis of a second substrate that is selected from phloretate or an analog thereof. The microbial cell further comprises a transferase enzyme forming FOPPA, or a derivative thereof, from the first substrate and the second substrate. As shown in FIG. 1, FOPPA is composed of a compound derived from farnesyl-PP or farnesol and phloretate.

In various embodiments, an analog of phloretate is produced. The analog of phloretate is selected from cinnamic acid, hydrocinnamic acid, and p-coumaric acid.

In various embodiments, the enzyme pathway for the synthesis of the first substrate comprises one or more farnesyl diphosphate synthases (FPPS). In some embodiments, the FPPS enzyme is a Saccharomyces cerevisiae farnesyl pyrophosphate synthase (ScFPPS), which comprises the amino acid sequence of SEQ ID NO: 1, or a derivative thereof. In various embodiments, the derivative comprises an amino acid sequence having at least about 50% identity to SEQ ID NO:1, or in other embodiments, at least about 60%, or at least about 70%, or at least about 80%, or at least about 90%, or at least about 95%, or at least about 97%, 98%, or 99% amino acid sequence identity to SEQ ID NO:1. Alternatively, the FPPS is E. coli ispA, or a variant thereof. Numerous alternative FPPS enzymes are known in the art, and may be employed for conversion of IPP and/or DMAPP to farnesyl diphosphate in accordance with this aspect.

In some embodiments, the FPPS comprises an amino acid sequence having from 1 to 20 amino acid modifications or having from 1 to 10 amino acid modifications with respect to SEQ ID NO: 1, the amino acid modifications being independently selected from amino acid substitutions, deletions, and insertions.

In various embodiments, the enzyme pathway for the synthesis of the first substrate comprises one or more overexpressed enzymes of the methylerythritol phosphate (MEP) pathway or mevalonic acid (MVA) pathway. In such embodiments, the MEP or MVA pathway is engineered to increase carbon flux to farnesyl diphosphate or farnesol.

The microbial cell will produce MEP or MVA products, which act as substrates for the enzyme pathway. The MEP (2-C-methyl-D-erythritol 4-phosphate) pathway, also called the MEP/DOXP (2-C-methyl-D-erythritol 4-phosphate/1-deoxy-D-xylulose 5-phosphate) pathway or the non-mevalonate pathway or the mevalonic acid-independent pathway refers to the pathway that converts glyceraldehyde-3-phosphate and pyruvate to IPP and DMAPP. The pathway, which is present in bacteria, typically involves action of the following enzymes: 1-deoxy-D-xylulose-5-phosphate synthase (Dxs), 1-deoxy-D-xylulose-5-phosphate reductoisomerase (IspC), 4-diphosphocytidyl-2-C-methyl-D-erythritol synthase (IspD), 4-diphosphocytidyl-2-C-methyl-D-erythritol kinase (IspE), 2C-methyl-D-erythritol 2,4-cyclodiphosphate synthase (IspF), 1-hydroxy-2-methyl-2-(E)-butenyl 4-diphosphate synthase (IspG), and isopentenyl diphosphate isomerase (IspH). The MEP pathway, and the genes and enzymes that make up the MEP pathway, are described in U.S. Pat. No. 8,512,988, which is hereby incorporated by reference in its entirety. For example, genes that make up the MEP pathway include dxs, ispC, ispD, ispE, ispF, ispG, ispH, idi, and ispA. In some embodiments, the host cell expresses or overexpresses one or more of dxs, ispC, ispD, ispE, ispF, ispG, ispH, idi, ispA, or modified variants thereof, which results in the increased production of IPP and DMAPP. In some embodiments, the FPP or farnesol is produced at least in part by metabolic flux through an MEP pathway, and wherein the host cell has at least one additional gene copy of one or more of dxs, ispC, ispD, ispE, ispF, ispG, ispH, idi, ispA, or modified variants thereof.

The MVA pathway refers to the biosynthetic pathway that converts acetyl-CoA to IPP. The mevalonate pathway, which will be present in yeast, typically comprises enzymes that catalyze the following steps: (a) condensing two molecules of acetyl-CoA to acetoacetyl-CoA (e.g., by action of acetoacetyl-CoA thiolase); (b) condensing acetoacetyl-CoA with acetyl-CoA to form hydroxymethylglutaryl-CoenzymeA (HMG-CoA) (e.g., by action of HMG-CoA synthase (HMGS)); (c) converting HMG-CoA to mevalonate (e.g., by action of HMG-CoA reductase (HMGR)); (d) phosphorylating mevalonate to mevalonate 5-phosphate (e.g., by action of mevalonate kinase (MK)); (e) converting mevalonate 5-phosphate to mevalonate 5-pyrophosphate (e.g., by action of phosphomevalonate kinase (PMK)); and (f) converting mevalonate 5-pyrophosphate to isopentenyl pyrophosphate (e.g., by action of mevalonate pyrophosphate decarboxylase (MPD)). The MVA pathway, and the genes and enzymes that make up the MVA pathway, are described in U.S. Pat. No. 7,667,017, which is hereby incorporated by reference in its entirety. In some embodiments, the host cell expresses or overexpresses one or more of acetoacetyl-CoA thiolase, HMGS, HMGR, MK. PMK, and MPD or modified variants thereof, which results in the increased production of IPP and DMAPP. In some embodiments. FPP or farnesol is produced at least in part by metabolic flux through an MV A pathway, and wherein the host cell has at least one additional gene copy of one or more of acetoacetyl-CoA thiolase, HMGS, HMGR. MK, PMK, MPD, or modified variants thereof.

In some embodiments, the host cell is a bacterial host cell engineered to increase production of IPP and DMAPP from glucose as described in US 2018/0245103 and US 2018/0216137, the contents of which are hereby incorporated by reference in their entireties. For example, in some embodiments the host cell overexpresses MEP pathway enzymes, with balanced expression to push/pull carbon flux to IPP and DMAP. In some embodiments, the host cell is engineered to increase the availability or activity of Fe—S cluster proteins, so as to support higher activity of IspG and IspH, which are Fe—S enzymes. In some embodiments, the host cell is engineered to overexpress IspG and IspH, so as to provide increased carbon flux to 1-hydroxy-2-methyl-2-(E)-butenyl 4-diphosphate (HMBPP) intermediate, but with balanced expression to prevent accumulation of HMBPP at an amount that reduces cell growth or viability, or at an amount that inhibits MEP pathway flux and/or terpenoid production. In some embodiments, the host cell exhibits higher activity of IspH relative to IspG. In some embodiments, the host cell is engineered to downregulate the ubiquinone biosynthesis pathway, e.g., by reducing the expression or activity of IspB, which uses IPP and FPP substrate.

In various embodiments, the enzyme pathway for the synthesis of the second substrate comprises tyrosine ammonia lyase (TAL) and phenolic acid reductase (PAR), or variants thereof. The enzyme pathway may further comprise phenylalanine ammonia lyase (PAL) and cinnamate-4-hydroxylase (C4H), or variants thereof.

In various embodiments, the enzyme pathway for the synthesis of the second substrate comprises the enzymes tyrosine ammonia lyase (TAL), phenylalanine ammonia lyase (PAL), cinnamate-4-hydroxylase (C4H), 4-coumarate-CoA ligase (4CL), hydroxycinnamoyl-CoA double bond reductase (HCDBR) and/or phenolic acid reductase (PAR), chalcone synthase (CHS), and phloretin hydrolase (PH).

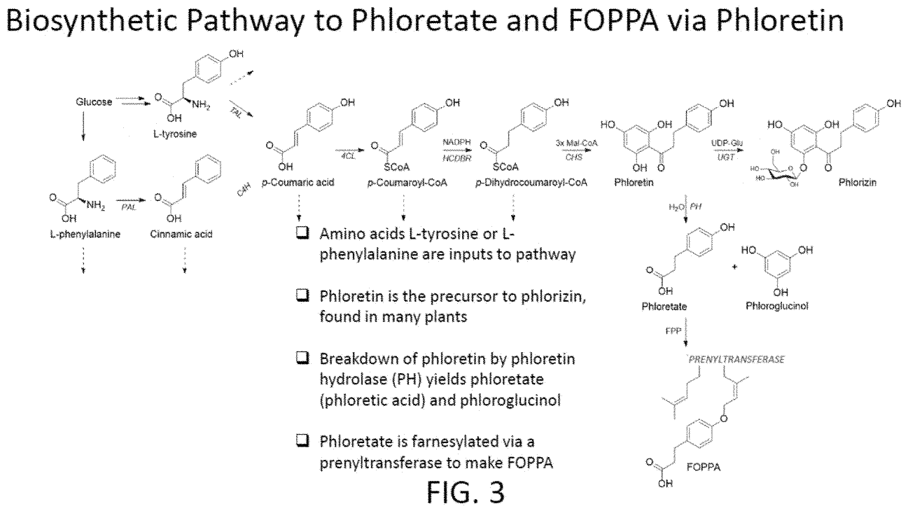

An exemplary enzyme pathway for the synthesis of a second substrate that uses one or more of TAL, PAL, C4H, PAR, 4CL, HCDBR, CHS, and PH is provided in the plant-derived biosynthetic pathway shown in FIG. 3. In this pathway, the amino acids L-tyrosine or L-phenylalanine are used as precursors. When L-phenylalanine is the precursor substrate, it is converted by a phenylalanine ammonia lyase (PAL) to cinnamic acid. Alternatively, L-tyrosine can be converted to p-Coumaric acid by a tyrosine ammonia lyase. The cinnamic acid is converted to p-coumaric acid by a cinnamate-4-hydroxylase (C4H). A 4-coumaric acid CoA ligase (4CL) converts p-coumaric acid top-coumaroyl-CoA, which is converted by hydroxycinnamoyl-CoA double bond reductase (HCDBR) and nicotinamide adenine dinucleotide phosphate (NADPH) to p-dihydrocoumaroyl-CoA. The p-dihydrocoumaroyl-CoA is converted to phloretin by chalcone synthase (CHS) and malonyl-CoA. Phloretin is broken down to phloretate and phloroglucinol by phloretin hydrolase (PH). Using farnesyl pyrophosphate, farnesyl-phosphate, or farnesol, the phloretate is farnesylated via a transferase, such as prenyltransferase, to create FOPPA.

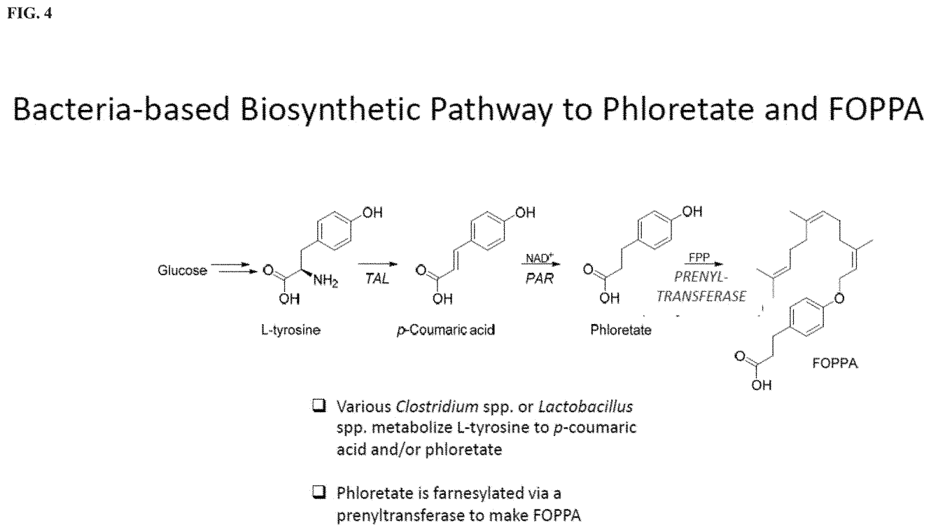

Yet another exemplary pathway for the synthesis of a second substrate is provided in the alternative biosynthetic pathway shown in FIG. 4. In this pathway, the amino acid L-tyrosine is the precursor substrate. The L-tyrosine is converted to p-coumaric acid by a tyrosine ammonia lyase (TAL). The p-coumaric acid is then converted to phloretate by a phenolic acid reductase (PAR) and NAD+ cofactor. With farnesyl pyrophosphate, farnesyl-phosphate, or farnesol, the phloretate is farnesylated via a transferase, such as prenyltransferase, to make FOPPA.

In various embodiments, the PAR enzyme comprises an amino acid sequence that has at least about 50% amino acid sequence identity with a wild type PAR enzyme from Clostridium spp., such as Clostridium orbiscindens, or Lactobacillus spp., such as Lactobacillus plantarum. In various embodiments, the PAR enzyme comprises an amino acid sequence having at least about 60% identity, or at least about 70% identity, or at least about 80% identity, or at least about 90% identity, or at least about 95% identity, or at least about 97%, 98%, or 99% amino acid sequence identity to PAR from Clostridium orbiscindens or Lactobacillus plantarum.

In various embodiments, the TAL enzyme comprises the amino acid sequence of a wild type TAL enzyme (or derivative thereof) from Rhodobacter spp., e.g., Rhodobacter sphaeroides; Rhodotorula spp., e.g., Rhodotorula glutinis; Herpatosiphon spp., e.g., Herpatosiphon auranticus; Flavobacterium spp., e.g., Flavobacterium johnsoniae; Saccharothrix spp., e.g., Saccharothrix espanaensis; Amaranthus spp., e.g. Amaranthus hypocondriacus; Amborella spp., e.g. Amborella trichopoda; Aquilegia spp., e.g. Aquilegia coerulea; Arabidopsis spp., e.g., Arabidopsis thaliana; Azadirachta spp., e.g. Azadiractha indica; Bambusa spp., e.g., Bambusa vulgaris; Beta spp., e.g. Beta vulgaris; Cannabis spp., e.g. Cannabis sativa; Capsicum spp., e.g., Capsicum annuum; Carica spp., e.g. Carica papaya Catharanthus spp., e.g., Catharanthus roseus; Cistanche spp., e.g., Cistanche deserticola; Citrus spp., e.g. Citrus sinensis; Cucumis spp., e.g. Cucumis melo; Elaeis spp., e.g., Elaeis guineensis; Eucalyptus spp., e.g. Eucalyptus grandis; Glycine spp., e.g. Glycine max; Gossypium spp., e.g. Gossypium Raimondi; Helianthus spp., e.g., Helianthus tuberosus; Kalanchoë spp., e.g. Kalanchoë fedtschenkoi; Linum spp., e.g. Linum usitatissimum; Malus spp., e.g. Malus×domestica; Manihot spp., e.g. Manihot esculenta; Mimulus spp., e.g. Mimulus guttatus; Musa spp., e.g. Musa acuminate; Nelumbo spp., e.g. Nelumbo nucifera; Nicotiana spp., e.g., Nicotiana tabacum; Oryza spp., e.g. Oryza sativa; Petroselinum spp., e.g., Petroselinum crispum; Phalaenopsis spp., e.g. Phalaenopsis equestris; Phyllostacys spp., e.g. Phyllostacys edulis; Physcomitrella spp., e.g., Physcomitrella patens; Pisum spp., e.g. Pisum sativum; Pinus spp., e.g. Pinus taeda; Populus spp., e.g., Populus trichocarpa; Selaginella spp., e.g. Selaginella moellendorfii; Sesamum spp., e.g. Sesamum indicum; Spirodela spp., e.g. Spirodela polyrhiza; Stevia spp., e.g. Stevia rebaudiana; Thapsia spp., e.g. Thapsia villosa; Triticum spp., e.g. Triticum aestivum; Utricularia spp., e.g. Utricularia gibba; Vigna spp., e.g., Vigna radiate; Vitis spp., e.g. Vitis vinifera or Zea spp., e.g. Zea mays. In various embodiments, the TAL enzyme comprises an amino acid sequence having at least about 60% identity, or at least about 70% identity, or at least about 80% identity, or at least about 900% identity, or at least about 95% identity, or at least about 97%, 98%, or 99% amino acid sequence identity to a TAL enzyme of a species disclosed in this paragraph.

In various embodiments, the PAL enzyme comprises an amino acid of a wild type PAL enzyme (or derivative thereof) from Brevibacillus spp., e.g., Brevibacillus laterosporus; Streptomyces spp.; Dictyostelium spp., e.g., Dictyostelium discoideum; Photorhabdus spp., e.g., Photorhabdus luminescens; Amaranthus spp., e.g. Amaranthus hypocondriacus; Amborella spp., e.g. Amborella trichopoda; Aquilegia spp., e.g. Aquilegia coerulea; Arabidopsis spp., e.g., Arabidopsis thaliana; Azadirachta spp., e.g. Azadiractha indica; Bambusa spp., e.g., Bambusa vulgaris; Beta spp., e.g. Beta vulgaris; Cannabis spp., e.g. Cannabis sativa; Capsicum spp., e.g., Capsicum annuum; Carica spp., e.g. Carica papaya; Catharanthus spp., e.g., Catharanthus roseus; Cistanche spp., e.g., Cistanche deserticola; Citrus spp., e.g. Citrus sinensis; Cucumis spp., e.g. Cucumis melo; Elaeis spp., e.g., Elaeis guineensis; Eucalyptus spp., e.g. Eucalyptus grandis; Glycine spp., e.g. Glycine max; Gossypium spp., e.g. Gossypium Raimondi; Helianthus spp., e.g., Helianthus tuberosus; Kalanchoë spp., e.g. Kalanchoë fedtschenkoi; Linum spp., e.g. Linum usitatissimum; Malus spp., e.g. Malus×domestica; Manihot spp., e.g. Manihot esculenta; Mimulus spp., e.g. Mimulus guttatus; Musa spp., e.g. Musa acuminate; Nelumbo spp., e.g. Nelumbo nucifera; Nicotiana spp., e.g., Nicotiana tabacum; Oryza spp., e.g. Oryza sativa; Petroselinum spp., e.g., Petroselinum crispum; Phalaenopsis spp., e.g. Phalaenopsis equestris; Phyllostacys spp., e.g. Phyllostacys edulis; Physcomitrella spp., e.g., Physcomitrella patens; Pisum spp., e.g. Pisum sativum; Pinus spp., e.g. Pinus taeda; Populus spp., e.g., Populus trichocarpa; Selaginella spp., e.g. Selaginella moellendorfii; Sesamum spp., e.g. Sesamum indicum; Spirodela spp., e.g. Spirodela polyrhiza; Stevia spp., e.g. Stevia rebaudiana; Thapsia spp., e.g. Thapsia villosa; Triticum spp., e.g. Triticum aestivum; Utricularia spp., e.g. Utricularia gibba; Vigna spp., e.g., Vigna radiate; Vitis spp., e.g. Vitis vinifera; or Zea spp., e.g. Zea mays. In various embodiments, the PAL enzyme comprises an amino acid sequence having at least about 60% identity, or at least about 70% identity, or at least about 80% identity, or at least about 90% identity, or at least about 95% identity, or at least about 97%, 98%, or 99% amino acid sequence identity to a PAL enzyme of a species disclosed in this paragraph.

In various embodiments, the C4H comprises an amino acid sequence of a wild type C4H enzyme (or derivative thereof) from Amaranthus spp., e.g. Amaranthus hypocondriacus; Amborella spp., e.g. Amborella trichopoda; Aquilegia spp., e.g. Aquilegia coerulea; Arabidopsis spp., e.g., Arabidopsis thaliana; Azadirachta spp., e.g. Azadiractha indica; Bambusa spp., e.g., Bambusa vulgaris; Beta spp., e.g. Beta vulgaris; Cannabis spp., e.g. Cannabis sativa; Capsicum spp., e.g., Capsicum annuum; Carica spp., e.g. Carica papaya; Catharanthus spp., e.g., Catharanthus roseus; Cistanche spp., e.g., Cistanche deserticola; Citrus spp., e.g. Citrus sinensis; Cucumis spp., e.g. Cucumis melo; Elaeis spp., e.g., Elaeis guineensis; Eucalyptus spp., e.g. Eucalyptus grandis; Glycine spp., e.g. Glycine max; Gossypium spp., e.g. Gossypium Raimondi; Helianthus spp., e.g., Helianthus tuberosus; Kalanchoë spp., e.g. Kalanchoë fedtschenkoi; Linum spp., e.g. Linum usitatissimum; Malus spp., e.g. Malus×domestica; Manihot spp., e.g. Manihot esculenta; Mimulus spp., e.g. Mimulus guttatus; Musa spp., e.g. Musa acuminate; Nelumbo spp., e.g. Nelumbo nucifera; Nicotiana spp., e.g., Nicotiana tabacum; Oryza spp., e.g. Oryza sativa; Petroselinum spp., e.g., Petroselinum crispum; Phalaenopsis spp., e.g. Phalaenopsis equestris; Phyllostacys spp., e.g. Phyllostacys edulis; Physcomitrella spp., e.g., Physcomitrella patens; Pisum spp., e.g. Pisum sativum; Pinus spp., e.g. Pinus taeda; Populus spp., e.g., Populus trichocarpa; Selaginella spp., e.g. Selaginella moellendorfii; Sesamum spp., e.g. Sesamum indicum; Spirodela spp., e.g. Spirodela polyrhiza; Stevia spp., e.g. Stevia rebaudiana; Thapsia spp., e.g. Thapsia villosa; Triticum spp., e.g. Triticum aestivum; Utricularia spp., e.g. Utricularia gibba; Vigna spp., e.g., Vigna radiate; Vitis spp., e.g. Vitis vinifera; or Zea spp., e.g. Zea mays. In various embodiments, the C4H enzyme comprises an amino acid sequence having at least about 60% identity, or at least about 70% identity, or at least about 80% identity, or at least about 90% identity, or at least about 95% identity, or at least about 97%, 98%, or 99% amino acid sequence identity to a C4H enzyme of a species disclosed in this paragraph.

In various embodiments, the 4CL enzyme comprises an amino acid sequence of a wild type 4CL enzyme (or derivative thereof) from Amaranthus spp., e.g. Amaranthus hypocondriacus; Amborella spp., e.g. Amborella trichopoda; Aquilegia spp., e.g. Aquilegia coerulea; Arabidopsis spp., e.g., Arabidopsis thaliana; Azadirachta spp., e.g. Azadiractha indica; Bambusa spp., e.g., Bambusa vulgaris; Beta spp., e.g. Beta vulgaris; Cannabis spp., e.g. Cannabis sativa; Capsicum spp., e.g., Capsicum annuum; Carica spp., e.g. Carica papaya; Catharanthus spp., e.g., Catharanthus roseus; Cistanche spp., e.g., Cistanche deserticola; Citrus spp., e.g. Citrus sinensis; Cucumis spp., e.g. Cucumis melo; Elaeis spp., e.g., Elaeis guineensis; Eucalyptus spp., e.g. Eucalyptus grandis; Glycine spp., e.g. Glycine max; Gossypium spp., e.g. Gossypium Raimondi; Helianthus spp., e.g., Helianthus tuberosus; Kalanchoë spp., e.g. Kalanchoë fedtschenkoi; Linum spp., e.g. Linum usitatissimum; Malus spp., e.g. Malus×domestica; Manihot spp., e.g. Manihot esculenta; Mimulus spp., e.g. Mimulus guttatus; Musa spp., e.g. Musa acuminate; Nelumbo spp., e.g. Nelumbo nucifera; Nicotiana spp., e.g., Nicotiana tabacum; Oryza spp., e.g. Oryza sativa; Petroselinum spp., e.g., Petroselinum crispum; Phalaenopsis spp., e.g. Phalaenopsis equestris; Phyllostacys spp., e.g. Phyllostacys edulis; Physcomitrella spp., e.g., Physcomitrella patens; Pisum spp., e.g. Pisum sativum; Pinus spp., e.g. Pinus taeda; Populus spp., e.g., Populus trichocarpa; Selaginella spp., e.g. Selaginella moellendorfii; Sesamum spp., e.g. Sesamum indicum; Spirodela spp., e.g. Spirodela polyrhiza; Stevia spp., e.g. Stevia rebaudiana; Thapsia spp., e.g. Thapsia villosa; Triticum spp., e.g. Triticum aestivum; Utricularia spp., e.g. Utricularia gibba; Vigna spp., e.g., Vigna radiate; Vitis spp., e.g. Vitis vinifera; or Zea spp., e.g. Zea mays. In various embodiments, the 4CL enzyme comprises an amino acid sequence having at least about 60% identity, or at least about 70% identity, or at least about 80% identity, or at least about 90% identity, or at least about 95% identity, or at least about 97%, 98%, or 99% amino acid sequence identity to a 4CL enzyme of a species disclosed in this paragraph.

In various embodiments, the HCDBR enzyme comprises an amino acid sequence of a wild type HCDBR enzyme (or derivative thereof) from Amaranthus spp., e.g. Amaranthus hypocondriacus; Amborella spp., e.g. Amborella trichopoda; Aquilegia spp., e.g. Aquilegia coerulea; Arabidopsis spp., e.g., Arabidopsis thaliana; Azadirachta spp., e.g. Azadiractha indica; Bambusa spp., e.g., Bambusa vulgaris; Beta spp., e.g. Beta vulgaris; Cannabis spp., e.g. Cannabis sativa; Capsicum spp., e.g., Capsicum annuum; Carica spp., e.g. Carica papaya; Catharanthus spp., e.g., Catharanthus roseus; Cistanche spp., e.g., Cistanche deserticola; Citrus spp., e.g. Citrus sinensis; Cucumis spp., e.g. Cucumis melo; Elaeis spp., e.g., Elaeis guineensis; Eucalyptus spp., e.g. Eucalyptus grandis; Glycine spp., e.g. Glycine max; Gossypium spp., e.g. Gossypium Raimondi; Helianthus spp., e.g., Helianthus tuberosus; Kalanchoë spp., e.g. Kalanchoë fedtschenkoi; Linum spp., e.g. Linum usitatissimum; Malus spp., e.g. Malus×domestica; Manihot spp., e.g. Manihot esculenta; Mimulus spp., e.g. Mimulus guttatus; Musa spp., e.g. Musa acuminate; Nelumbo spp., e.g. Nelumbo nucifera; Nicotiana spp., e.g., Nicotiana tabacum; Oryza spp., e.g. Oryza sativa Petroselinum spp., e.g., Petroselinum crispum; Phalaenopsis spp., e.g. Phalaenopsis equestris; Phyllostacys spp., e.g. Phyllostacys edulis Physcomitrella spp., e.g., Physcomitrella patens; Pisum spp., e.g. Pisum sativum; Pinus spp., e.g. Pinus taeda; Populus spp., e.g., Populus trichocarpa Selaginella spp., e.g. Selaginella moellendorfii; Sesamum spp., e.g. Sesamum indicum; Spirodela spp., e.g. Spirodela polyrhiza; Stevia spp., e.g. Stevia rebaudiana; Thapsia spp., e.g. Thapsia villosa; Triticum spp., e.g. Triticum aestivum; Utricularia spp., e.g. Utricularia gibba; Vigna spp., e.g., Vigna radiate; Vitis spp., e.g. Vitis vinifera; or Zea spp., e.g. Zea mays. In various embodiments, the HCDBR enzyme comprises an amino acid sequence having at least about 60% identity, or at least about 70% identity, or at least about 80% identity, or at least about 90% identity, or at least about 95% identity, or at least about 97%, 98%, or 99% amino acid sequence identity to a HCDBR enzyme of a species disclosed in this paragraph.

In various embodiments, the CHS enzyme comprises an amino acid sequence of a wild type CHS enzyme (or derivative thereof) from Amaranthus spp., e.g. Amaranthus hypocondriacus; Amborella spp., e.g. Amborella trichopoda; Aquilegia spp., e.g. Aquilegia coerulea; Arabidopsis spp., e.g., Arabidopsis thaliana; Azadirachta spp., e.g. Azadiractha indica; Bambusa spp., e.g., Bambusa vulgaris; Beta spp., e.g. Beta vulgaris; Cannabis spp., e.g. Cannabis sativa; Capsicum spp., e.g., Capsicum annuum; Carica spp., e.g. Carica papaya; Catharanthus spp., e.g., Catharanthus roseus; Cistanche spp., e.g., Cistanche deserticola; Citrus spp., e.g. Citrus sinensis; Cucumis spp., e.g. Cucumis melo; Elaeis spp., e.g., Elaeis guineensis; Eucalyptus spp., e.g. Eucalyptus grandis; Glycine spp., e.g. Glycine max; Gossypium spp., e.g. Gossypium Raimondi; Helianthus spp., e.g., Helianthus tuberosus; Kalanchoë spp., e.g. Kalanchoë fedtschenkoi; Linum spp., e.g. Linum usitatissimum; Malus spp., e.g. Malus×domestica; Manihot spp., e.g. Manihot esculenta; Mimulus spp., e.g. Mimulus guttatus; Musa spp., e.g. Musa acuminate; Nelumbo spp., e.g. Nelumbo nucifera; Nicotiana spp., e.g., Nicotiana tabacum; Oryza spp., e.g. Oryza sativa; Petroselinum spp., e.g., Petroselinum crispum; Phalaenopsis spp., e.g. Phalaenopsis equestris; Phyllostacys spp., e.g. Phyllostacys edulis; Physcomitrella spp., e.g., Physcomitrella patens; Pisum spp., e.g. Pisum sativum; Pinus spp., e.g. Pinus taeda; Populus spp., e.g., Populus trichocarpa; Selaginella spp., e.g. Selaginella moellendorfii; Sesamum spp., e.g. Sesamum indicum; Spirodela spp., e.g. Spirodela polyrhiza; Stevia spp., e.g. Stevia rebaudiana; Thapsia spp., e.g. Thapsia villosa; Triticum spp., e.g. Triticum aestivum; Utricularia spp., e.g. Utricularia gibba; Vigna spp., e.g., Vigna radiate; Vitis spp., e.g. Vitis vinifera; or Zea spp., e.g. Zea mays. In various embodiments, the CHS enzyme comprises an amino acid sequence having at least about 60% identity, or at least about 70% identity, or at least about 80% identity, or at least about 90% identity, or at least about 95% identity, or at least about 97%, 98%, or 99% amino acid sequence identity to a CHS enzyme of a species disclosed in this paragraph.

In various embodiments, the PH enzyme comprises an amino acid sequence of a wild type PH enzyme (or derivative thereof) from Acidaminococcus spp., e.g. Acidaminococcus fermentans strain ATCC 25085; Anaerovibrio spp., e.g. Anaerovibrio lipolyticus; Aspergillus spp., e.g. Aspergillus nidulans; Butyricicoccus spp., e.g. Butyricicoccus pullicaecorum; Canis spp., e.g. Canis lupus; Clostridium spp., e.g. Clostridium aurantibutyricum; Dialister spp., e.g. Dialister succinatiphilus; Erwinia spp., e.g. Erwinia herbicola; Eubacterium spp., e.g. Eubacterium ramulus; Flavonifractor spp., e.g. Flavonifractor sp. An112 Homo spp., e.g. Homo sapiens; Lachnospira spp., e.g. Lachnospira multipara; Megasphaera spp., e.g. Megasphaera elsdenii; Mus spp., e.g. Mus musculus; Oribacterium spp., e.g. Oribacterium sp. P6A1; Oryctolagus spp., e.g. Orvctolagus cuniculus; Pantoea spp., e.g. Pantoea agglomerans. Parasporobacterum spp., e.g. Parasporobacterium paucivorans; Propionispira spp., e.g. Propionispira arboris; Ratus spp., e.g. Ratus norvegicus; Roseburia spp., e.g. Roseburia sp. CAG:50; Selenomonas spp., e.g. Selenomonas ruminantium; or Sharpea spp., e.g. Sharpea azabuensis. In various embodiments, the PH enzyme comprises an amino acid sequence having at least about 60% identity, or at least about 70% identity, or at least about 80% identity, or at least about 90% identity, or at least about 95% identity, or at least about 97%, 98%, or 99% amino acid sequence identity to a PH enzyme of a species disclosed in this paragraph.

In some embodiments, the enzyme pathway for the synthesis of the second substrate may comprise one or more cytochrome P450 reductases (CPR). In some embodiments, the CPR comprises an amino acid sequence identity with a wild type CPR from Saccharomyces spp., e.g. Saccharomyces cerevisiae; Amaranthus spp., e.g. Amaranthus hypocondriacus; Amborella spp., e.g. Amborella trichopoda; Aquilegia spp., e.g. Aquilegia coerulea; Arabidopsis spp., e.g., Arabidopsis thaliana; Azadirachta spp., e.g. Azadiractha indica; Bambusa spp., e.g., Bambusa vulgaris; Beta spp., e.g. Beta vulgaris; Cannabis spp., e.g. Cannabis sativa; Capsicum spp., e.g., Capsicum annuum; Carica spp., e.g. Carica papaya; Catharanthus spp., e.g., Catharanthus roseus; Cistanche spp., e.g., Cistanche deserticola; Citrus spp., e.g. Citrus sinensis; Cucumis spp., e.g. Cucumis melo; Elaeis spp., e.g., Elaeis guineensis; Eucalyptus spp., e.g. Eucalyptus grandis; Glycine spp., e.g. Glycine max; Gossypium spp., e.g. Gossypium Raimondi; Helianthus spp., e.g., Helianthus tuberosus; Kalanchoë spp., e.g. Kalanchoë fedtschenkoi; Linum spp., e.g. Linum usitatissimum; Malus spp., e.g. Malus×domestica; Manihot spp., e.g. Manihot esculenta; Mimulus spp., e.g. Mimulus guttatus; Musa spp., e.g. Musa acuminate; Nelumbo spp., e.g. Nelumbo nucifera; Nicotiana spp., e.g., Nicotiana tabacum; Oryza spp., e.g. Oryza sativa; Petroselinum spp., e.g., Petroselinum crispum; Phalaenopsis spp., e.g. Phalaenopsis equestris; Phyllostacys spp., e.g. Phyllostacys edulis; Physcomitrella spp., e.g., Physcomitrella patens; Pisum spp., e.g. Pisum sativum; Pinus spp., e.g. Pinus taeda; Populus spp., e.g., Populus trichocarpa; Selaginella spp., e.g. Selaginella moellendorfii; Sesamum spp., e.g. Sesamum indicum; Spirodela spp., e.g. Spirodela polyrhiza; Stevia spp., e.g. Stevia rebaudiana; Thapsia spp., e.g. Thapsia villosa; Triticum spp., e.g. Triticum aestivum; Utricularia spp., e.g. Utricularia gibba; Vigna spp., e.g., Vigna radiate; Vitis spp., e.g. Vitis vinifera; or Zea spp., e.g. Zea mays. In various embodiments, the CPR enzyme comprises an amino acid sequence having at least about 60% identity, or at least about 70% identity, or at least about 80% identity, or at least about 90% identity, or at least about 95% identity, or at least about 97%, 98%, or 99% amino acid sequence identity to a CPR enzyme of a species disclosed in this paragraph.

In various embodiments, the enzyme pathway for the synthesis of the second substrate comprises an enzyme, a pathway, and/or reaction that converts p-coumaric acid to phloretate in Lactobacillus plantarum. Exemplary enzymes, pathways, and reactions are disclosed in Barthelmebs et al., Applied and Environmental Microbiology, 66(8): 3368-75 (August 2000), the contents of which are hereby incorporated by reference in their entirety.

In various embodiments, the enzyme pathway for the synthesis of the second substrate comprises an enzyme, a pathway, and/or reaction for the production of phloretate from tyrosine by Clostridium spp. Exemplary enzymes, pathways, and reactions are disclosed in Mead, G., Journal of General Microbiology, 67: 47-56 (1971); Elsden et al., Arch. Microbiol., 107: 283-88 (1976); and/or Jellet et al., Can. J. Microbiol., 26: 448-53 (1980), the contents of which are hereby incorporated by reference in their entireties.

In various embodiments, the enzyme pathway for the synthesis of the second substrate comprises an enzyme, a pathway, and/or reaction for the production of phloretate by Clostridium orbiscindens. Exemplary enzymes, pathways, and reactions are disclosed in Steed et al., Science. 357: 498-502 (Aug. 4, 2017), the contents of which are hereby incorporated by reference in their entirety.

In various embodiments, the enzyme pathway for the synthesis of the second substrate comprises an enzyme, a pathway, and/or reaction disclosed in PCT Pub. No. WO 2016/193504, the contents of which are hereby incorporated by reference in their entirety.

In various embodiments, the transferase enzyme comprises an amino acid sequence of a Aspergillus terreus aromatic Prenyl Transferase (AtaPT) enzyme having an accession number selected from KP893683, EAU39348, EAU39467, EAU36097, EAU36020, EAU31601, EAU29429, EAU29303 and a variant thereof. Examples of such a transferase enzyme are disclosed in Chen et al., Nature Chemical Biology, 13(2): 226-34 (Dec. 19, 2016), the contents of which are hereby incorporated by reference in their entirety. In some embodiments, the transferase enzyme comprises an amino acid sequence having at least about 60% identity, or at least about 70% identity, or at least about 80% identity, or at least about 90% identity, or at least about 95% identity, or at least about 97%, 98%, or 99% amino acid sequence identity with any one of the AtaPT enzymes having the accession number selected from KP893683, EAU39348, EAU39467, EAU36097, EAU36020, EAU31601, EAU29429, and EAU29303.

In various embodiments, the transferase enzyme comprises an amino acid sequence selected from SEQ ID NOs: 2-22, or a variant thereof. In some embodiments, the transferase enzyme comprises an amino acid sequence that has at least about 60% identity, or at least about 70% identity, or at least about 80% identity, or at least about 90% identity, or at least about 95% identity, or at least about 97%, 98%, or 99% amino acid sequence identity with any of SEQ ID NOs: 2-22.

In various embodiments, the transferase enzyme comprises the amino acid sequence of SEQ ID NO: 2 with one or more of the following modifications: deletion of amino acids corresponding to amino acids 1-10 of SEQ ID NO: 2 and a substitution at a position corresponding to H88, E91, S177, or W397 of SEQ ID NO: 2. In some embodiments, the transferase comprises a substitution selected from H88A, E91A, E91Q, E91D, S177A, and W397A.

In various embodiments, the transferase enzyme comprises the amino acid sequence of SEQ ID NO: 3 with one or more substitutions at positions corresponding to W97, E123, F170, A173, and F189 of SEQ ID NO: 3. In some embodiments, the transferase enzyme comprises a substitution selected from W97Y and A173M.

In various embodiments, the transferase enzyme comprises the amino acid sequence of SEQ ID NO: 4 with one or more substitutions at positions corresponding to Y80, W157, and M159 of SEQ ID NO: 4. In some embodiments, the transferase enzyme comprises a substitution selected from Y80W and M159A.

In various embodiments, at least one enzyme is a circular permutant. Circular permutant strategies for engineering enzymes are described in WO 2016/073740, which is hereby incorporated by reference in its entirety.

In various embodiments, the derivative of FOPPA is selected from 3-(4-farnesyloxyphenyl)-propionic acid methyl ester, 4-farnesyloxycinnamic acid methyl ester, and 4-farnesyloxycinnamic acid. Exemplary FOPPA derivatives are disclosed in in U.S. Pat. Nos. 4,939,171 and 9,814,659, US Publication No. 2011/0318439, and PCT Publication No. WO 2016/193501, the contents of which are hereby incorporated by reference in their entireties.

In various embodiments, the microbial cell is prokaryotic or eukaryotic. In some embodiments the microbial cell is a bacteria cell. In some embodiments, the microbial cell is a yeast cell. In some embodiments, the microbial host cell is a bacteria selected from Escherichia spp., Bacillus spp., Corynebacterium spp., Rhodobacter spp., Zymomonas spp., Vibrio spp., and Pseudomonas spp. For example, in some embodiments, the bacterial host cell is a species selected from Escherichia coli, Bacillus subtilis, Corynebacterium glutamicum, Rhodobacter capsulatus, Rhodobacter sphaeroides, Zymomonas mobilis, Vibrio natriegens, or Pseudomonas putida. In some embodiments, the bacterial host cell is E. coli. Alternatively, the microbial cell may be a yeast cell, such as but not limited to a species of Saccharomyces, Pichia, or Yarrowia, including Saccharomyces cerevisiae, Pichia pastoris, and Yarrowia lipolytica.

In some aspects, the invention provides a method for making FOPPA, or a derivative thereof, comprising: culturing the microbial cell as discussed herein, and recovering FOPPA, or a derivative thereof, from the cells or from the culture.

In some aspects, the invention provides a method for making FOPPA, or a derivative thereof, comprising: contacting a first substrate and a second substrate with a prenyltransferase to make FOPPA, or a derivative thereof, wherein the first substrate is selected from farnesyl pyrophosphate, farnesyl-phosphate, or farnesol; wherein the second substrate is selected from phloretate or a precursor or analog thereof. In some embodiments, the prenyltransferase is selected from Aspergillus terreus aromatic Prenyl Transferase (AtaPT) enzyme having an accession number selected from KP893683, EAU39348, EAU39467, EAU36097, EAU36020, EAU31601, EAU29429, and EAU29303, a variant thereof, or is selected from a transferase enzyme comprising an amino acid sequence selected from SEQ ID NOs: 2-22, or a variant thereof. Exemplary prenyltransferases are disclosed in Chen et al., Nature Chemical Biology, 13(2): 226-34 (Dec. 19, 2016), the contents of which are hereby incorporated by reference in their entirety. In various embodiments, the precursor or analog of phloretate is selected from cinnamic acid, hydrocinnamic acid, and p-coumaric acid.

In various embodiments, the prenyltransferase enzyme comprises an amino acid sequence having at least about 60% identity, or at least about 70% identity, or at least about 80% identity, or at least about 90% identity, or at least about 95% identity, or at least about 97%, 98%, or 99% amino acid sequence identity with any one of the enzymes having the accession number selected from KP893683, EAU39348, EAU39467, EAU36097, EAU36020, EAU31601, EAU29429, and EAU29303.

In various embodiments, the prenyltransferase enzyme comprises an amino acid sequence having has at least about 60% identity, or at least about 70% identity, or at least about 80% identity, or at least about 90% identity, or at least about 95% identity, or at least about 97%, 98%, or 99% amino acid sequence identity with any one of SEQ ID NOs: 2-22.

In various embodiments, the prenyltransferase enzyme comprises the amino acid sequence of SEQ ID NO: 2 with one or more of the following modifications: deletion of amino acids corresponding to amino acids 1-10 of SEQ ID NO: 2 and a substitution at a position corresponding to H88, E91, S177, or W397 of SEQ ID NO: 2. In some embodiments, the prenyltransferase comprises a substitution selected from H88A, E91A, E91Q, E91D, S177A, and W397A.

In various embodiments, the prenyltransferase enzyme comprises the amino acid sequence of SEQ ID NO: 3 with one or more substitutions at positions corresponding to W97, E123, F170, A173, and F189 of SEQ ID NO: 3. In some embodiments, the prenyltransferase enzyme comprises a substitution selected from W97Y and A173M.

In various embodiments, the prenyltransferase enzyme comprises the amino acid sequence of SEQ ID NO: 4 with one or more substitutions at positions corresponding to Y80, W157, and M159 of SEQ ID NO: 4. In some embodiments, the prenyltransferase enzyme comprises a substitution selected from Y80W and M159A.

In various embodiments, the prenyltransferase is expressed in a microbe and contacted with the first substrate and the second substrate in the form of whole cells expressing the prenyltransferase, cellular extract, or in purified form.

In various embodiments, the prenyltransferase is expressed in a microbe, wherein the microbe overexpresses an enzyme in the pathway for the synthesis of the first substrate.

In various embodiments, the phloretate or an analog thereof is fed to the culture or reaction.

In various embodiments, the phloretate, or a derivative thereof, is prepared from a phloretate precursor selected from L-phenylalanine, cinnamic acid, tyrosine, p-coumaric acid, p-coumaroyl-CoA, p-dihydrocoumaroyl-CoA, phloretin, p-hydroxyphenylpyruvic acid, and p-hydroxyphenyllactic acid by a reaction with one or more enzymes for producing the phloretate or a derivative thereof (as described herein). In some embodiments, the contacting of the phloretate, or a derivative thereof, and the farnesyl pyrophosphate, farnesyl-phosphate, and/or farnesol with a prenyltransferase occurs in a cell free system. In some embodiments, the prenyltransferase and/or the farnesyl pyrophosphate, farnesyl-phosphate, or farnesol are provided in the form of a cellular extract. In some embodiments, the cellular extract is an extract of a microbe overexpressing the prenyltransferase, and optionally overexpressing an enzyme to increase production of farnesyl pyrophosphate, farnesyl-phosphate, or farnesol.

In various embodiments, the farnesyl pyrophosphate, farnesyl-phosphate, and/or farnesol are provided in a cell free system comprising the prenyltransferase and at least one microbial cell engineered to produce the phloretate, or a derivative thereof.

In various embodiments, the phloretate is prepared from a precursor through an enzymatic pathway disclosed herein.

In various embodiments, the method further comprises harvesting the FOPPA from the cell culture or reaction.

In some aspects, the invention provides methods for making a product comprising FOPPA, or a derivative thereof, comprising producing FOPPA, or a derivative thereof, according to a method discussed above, and incorporating the FOPPA, or a derivative thereof, into the product. In some embodiments, the product is a skin-lightening composition. In other embodiments, the product is an anti-seborrheic composition.

In various embodiments, the product is a composition for use in an application selected from antioxidant, antibacterial, anthelmintic, anti-inflammatory, cancer chemopreventative, food additive, and fragrance component. The product may be a cosmetic composition, a pharmaceutical composition, or a nutraceutical composition. Exemplary applications are disclosed in US Pub. No. 2011/0318439, the contents of which are hereby incorporated by reference in their entirety.

All cited references are herein expressly incorporated by reference in their entirety.

SEQUENCE LISTING
>Saccharomyces cerevisiae farnesyl pyrophosphate synthase
(ScFPPS):
SEQ ID NO: 1
MASEKEIRRERFLNVFPKLVEELNASLLAYGMPKEACDWYAHSLNYNTPGGKLNRGLSVVDTY
AILSNKTVEQLGQEEYEKVAILGWCIELLQAYFLVADDMMDKSITRRGQPCWYKVPEVGEIAI
NDAFMLEAAIYKLLKSHFRNEKYYIDITELFHEVTFQTELGQLMDLITAPEDKVDLSKFSLKK
HSFIVTFKTAYYSFYLPVALAMYVAGITDEKDLKQARDVLIPLGEYFQIQDDYLDCFGTPEQI
GKIGTDIQDNKCSWVINKALELASAEQRKTLDENYGKEDSVAEAKCKKIFNDLKIEQLYHEYE
ESIAKDLKAKISQVDESRGFKADVLTAFLNKVYKRSK
>AtaPT (Aspergillus terreus aromatic Prenyl Transferase,
AMB20850.1)
SEQ ID NO: 2
MLPPSDSKDPRPWQILSQALGFPNYDQELWWQNTAETLNRVLEQCDYSVHLQYKYLAFYHKYI
LPSLGPFRRPGVEPEYISGLSHGGHPLEISVKIDKSKTICRLGLQAIGPLAGTARDPLNSFGD
RELLKNLATLLPHVDLRLFDHFNAQVGLDRAQCAVATTKLIKESHNIVCTSLDLKDGEVIPKV
YFSTIPKGLVTETPLFDLTFAAIEQMEVYHKDAPLRTALSSLKDFLRPRVPTDASITPPLTGL
IGVDCIDPMLSRLKVYLATFRMDLSLIRDYWTLGGLLTDAGTMKGLEMVETLAKTLKLGDEAC
ETLDAERLPFGINYAMKPGTAELAPPQIYFPLLGINDGFIADALVEFFQYMGWEDQANRYKDE
LKAKFPNVDISQTKNVHRWLGVAYSETKGPSMNIYYDVVAGNVARV
>TleC (Streptomyces blastmyceticus tryptophan
dimethylallyltransferase, BAP27943.1)
SEQ ID NO: 3
MESAGPGTGPQPPRTSGDFTPDTGVIAEMTGRPMRFDSDRYRPTDTYAEVACDKVCRAYEGLG
ADGGDRESLLAFLRDLTDPWGELPVGTPPEDACWVSIDGMPLETSVAWAGRKAGVRLSLESPR
GPAKRRMEDGMALTRRLAGRPGVSVDPCLRVEDLFTDDDPQGYFTIAHAVAWTPGGHPRYKIF
LNPAVRGREQAAARTEEAMIRLGLEQPWRALTEHLGGAYGPEHEPAALAMDLVPGDDFRVQVY
LAHSGVSAEAIDAKSAVAADHVPGSFARALRGINGADDTPEWKRKPPVTAFSFGPGRAVPGAT
LYVPMIPVHGSDAAARDRVAAFLRSEGMDAVGYEAVLDAISDRSLPESHTQNFISYRGGDSPR
FSVYLAPGVYREA
>MpnD (Marinactinospora thermotolerans aromatic
prenyltransferase, AFO85455.1)
SEQ ID NO: 4
MAGDPFVDNGTVSSQRPLRAVPGRYPPGATHLDAAVDTLVRCHAALGRAPSEAEAAVCLLRRL
WGRWGNTPVERPGWRSYVAVDGSPFELSAAWNGDGPAEVRVTVEATADPPTPEGNQEAGWEYL
RGLSRHPGAATARVLALEDLFRPQTPHDRCWIMHGMASRPGADPLFKVYLDPDARGAAEAPSV
LDEAMDRLGVRAAWQGLRGWLDEHGGSGRIGSLALDLADTDDARVKVYVQHAGLDWADIDRQA
AVARGHVPGAFSAALEEITGTEVPPHKPPVTCFAFHRGVGVPTAATLYIPMPAGVPESDARRR
SAAFMRRSGLDSAAYLAFLAAATGDGEGVRALQNFVAYRPAAPGGRPRFACYVAPGLYR
>PfIACE (Pestalotiopsis fici prenyltransferase, APC57597.1)
SEQ ID NO: 5
MAISTPSNGVSHVAKPLPNLKEVNKGIETDSEDRAFWWGALSEPLASLLEANHYTKEVQLHYL
RWFYQWILPALGPRPLDGKPYYGSWITHDLSPFEYSLNWKEKSSKQTIRFTIEAVTKQSGTAS
DPINQLGAKEFLEAVSKDVPGMDLTRFNQFLEATNVPNDCVDDAIAKHPAHFPRSRVWIAFDL
EHSGNLMAKSYFLPHWRAIQSGISANTIIGDTVKECNKADGSSYDGSLNAIESYLATFTRPEE
APQMGLLSNDCVAETPGSRLKVYFRSSADTLAKAKDMYNLGGRLKGPKMDASLKGISDFWYHL
FGLDSSDPASDDKVCIGNHKCIFVYEMRSSQGSEPDIDVKFHIPMWQLGKTDGQISELLASWF
ESHGHPDLASRYKSDLGTAFPKHNITGKSVGTHTYISITHTPKTGLYMTMYLSPKLPEFYY
>NphB (Streptomyces sp. CNZ306 aromatic prenyltransferase,
PJJ47653.1)
SEQ ID NO: 6
MIGIDFLECLVSEGIEAEGLYSAIEESARMVDAPFSRDKVWPILSAFGGGFSDAGGVIFSLQA
GKDVPEMEYSAQISAEVGDPYAHALATGVLNETDHPVSTVLAEIVSLAPTSEHYIDCGIVGGF
KKIYANFPHDQQKVSRLADLPAMPRAVGANAEFFDRYGLDNVALIGVDYRNKTINLYFQAPAE
TAGNLDPKTVSAMLRETGMSTPSEEMVAYADRAYRIYATLGWDSPEVMRLAFAPQPRRSIDLA
ELPARLEPRIEQFMRATPHKYPGALINATAAKWSKKHEVLDLAAYYQVSALHLKAIQAEEGQS
S
>ScFLT (Streptomyces cinnamonensis flaviolin linalyltranferase,
A2AXG5.1)
SEQ ID NO: 7
MMSGTADLAGVYAAVEESAGLLDVSCAREKVWPILAAFEDVLPTAVIAFRVATNARHEGEFDC
RFTVPGSIDPYAVALDKGLTHRSGHPIETLNADVQKHCAVDSYGVDFGVVGGFKKIWVYFPGG
RHESLAHLGEIPSMPPGLAATEGFFARYGLADKVDLIGVDYASKTMNVYFAASPEVVSAPTVL
AMHREIGLPDPSEQMLDFCSRAFGVYTTLNWDSSKVERIAYSVKTEDPLELSARLGSKVEQFL
KSVPYGIDTPKMVYAAVTAGGEEYYKLQSYYQWRTDSRLNLSYIGGRS
>StrFBP (Streptomyces sp. KO-3988 furaquinocin biosynthesis
prenyltransferase, Q2L6E3.1)
SEQ ID NO: 8
MPGTDDVAVDVASVYSAIEKSAGLLDVTAAREVVWPVLTAFEDVLEQAVIAFRVATNARHEGD
FDVRFTVPEEVDPYAVALSRSLIAKTDHPVGSLLSDIQQLCSVDTYGVDLGVKSGFKKVWVYF
PAGEHETLARLTGLTSMPGSLAGNVDFFTRYGLADKVDVIGIDYRSRTMNVYFAAPSECFERE
TVLAMHRDIGLPSPSEQMFKFCENSFGLYTTLNWDTMEIERISYGVKTENPMTFFARLGTRVE
HFVKNVPYGVDTQKMVYAAVTSSGEEYYKLQSYYRWRSVSRLNAAYIAARDKEST
>BrePT (Aspergillus versicolor brevianamide F reverse
prenyltransferase, AFM09725.1)
SEQ ID NO: 9
MTAPELRAPAGHPQEPPARSSPAQALSSYHHFPTSDQERWYQETGSLCSRFLEAGQYGLHQQY
QFMFFFMHHLIPALGPYPQKWRSTISRSGLPIEFSLNFQKGSHRLLRIGFEPVNFLSGSSQDP
FNRIPIADLLAQLARLQLRGFDTQCFQQLLTRFQLSLDEVRQLPPDDQPLKSQGAFGFDFNPD
GAILVKGYVFPYLKAKAAGVPVATLIAESVRAIDADRNQFMHAFSLINDYMQESTGYNEYTFL
SCDLVEMSRQRVKIYGAHTEVTWAKIAEMWTLGGRLIEEPEIMEGLARLKQIWSLLQIGEGSR
AFKGGFDYGKASATDQIPSPIIWNYEISPGSSFPVPKFYLPVHGENDLRVARSLAQFWDSLGW
SEHACAYPDMLQQLYPDLDVSRTSRLQSWISYSYTAKKGVYMSVYFHSQSTYLWEED
>7-DMATS (Aspergillus fumigatus At293 7-dimethylallyltrytophan
synthase, Q4WYG3.2)
SEQ ID NO: 10
MSIGAEIDSLVPAPPGLNGTAAGYPAKTQKELSNGDFDAHDGLSLAQLTPYDVLTAALPLPAP
ASSTGFWWRETGPVMSKLLAKANYPLYTHYKYLMLYHTHILPLLGPRPPLENSTHPSPSNAPW
RSFLTDDFTPLEPSWNVNGNSEAQSTIRLGIEPIGFEAGAAADPFNQAAVTQFMHSYEATEVG
ATLTLFEHFRNDMFVGPETYAALRAKIPEGEHTTQSFLAFDLDAGRVTTKAYFFPILMSLKTG
QSTTKVVSDSILHLALKSEVWGVQTIAAMSVMEAWIGSYGGAAKTEMISVDCVNEADSRIKIY
VRMPHTSLRKVKEAYCLGGRLTDENTKEGLKLLDELWRTVFGIDDEDAELPQNSHRTAGTIFN
FELPRGKWFPEPKVYLPVRHYCESDMQIASRLQTFFGRLGWHNMEKDYCKHLEDLFPHHPLSS
STGTHTFLSFSYKKQKGVYMTMYYNLRVYST
>CdpNPT (Aspergillus fumigatus cyclic dipeptide N-
prenyltransferase, ABR14712.1)
SEQ ID NO: 11
MDGEMTASPPDISACDTSAVDEQTGQSGQSQAPIPKDIAYHTLTKALLFPDIDQYQHWHHVAP
MLAKMLVDGKYSIHQQYEYLCLFAQLVAPVLGPYPSPGRDVYRCTLGGNMTVELSQNFQRSGS
TTRIAFEPVRYQASVGHDRFNRTSVNAFFSQLQLLVKSVNIELHHLLSEHLTLTAKDERNLNE
EQLTKYLTNFQVKTQYVVALDLRKTGIVAKEYFFPGIKCAATGQTGSNACFGAIRAVDKDGHL
DSLCQLIEAHFQQSKIDDAFLCCDLVDPAHTRFKVYIADPLVTLARAEEHWTLGGRLTDEDAA
VGLEIIRGLWSELGIIQGPLEPSAMMEKGLLPIMLNYEMKAGQRLPKPKLYMPLTGIPETKIA
RIMTAFFQRHDMPEQAEVFMENLQAYYEGKNLEEATRYQAWLSFAYTKEKGPYLSIYYFWPE
>BAE61387 (Aspergillus oryzae RIB40 putative prenyltransferase,
BAE61387.1)
SEQ ID NO: 12
MSLRNDLDNGRPTKRLESWDIASMWLSDRKDEIQDWWDFSGPQLATLAHEAGYSTMTQIELLL
FFRSVVLPRMGRFPDACRPRACAQSRSILTYDGSPIEYSWKWNNSANDHPEIRFCVEPVGDGL
CADGIVGGKLRATDEILVQLAKRVPSTDLEWYHHFRDSFGLGHWTDGPLHEDAGTWQVRRPRM
PVAFEFTPKGIVTKVYFTPPATLDDMPSFNMFADVVRPIGDKDTTALDESMEYLSRDPVGATL
RPDVLAIDCISPLKSRIKLYAGTAMTTFTSAISVLTLGGRIPVTRHSIDEMWALFRMVLGLHD
KFLQDEELPVQNPFQPSRAHPEDYYSGLLYYFNLAPGALLPDVKLYLPVIRYGRSDADIALGL
QRFMASRHRGQYVDGFQRAMEIISQRHKSGNGHRIQTYIACSFDKDGSLSLTSYLNPGVYFSS
ETVDV
>EAU34068 (Aspergillus terreus NIH2624 putative
prenyltransferase, EAU34068.1)
SEQ ID NO: 13
MLPPSDSKDPRPWQILSQALGFPNYDQELWWQNTAETLNRVLEQCDYSVHLQYKYLAFYHKYI
LPSLGPFRRPGVEPEYISGLSHGGHPLEISVKIDKSKTICRLGLQAIGPLAGTARDPLNSFGD
RELLKNLATLLPHVDLRLFDHFNAQVGLDRAQCAVATTKLIKESHNIVCTSLDLKDGEVIPKV
YFSTIPKGLVTETPLFDLTFAAIEQMEVYHKDAPLRTALSSLKDFLRPRVPTDASITPPLTGL
IGVDCIDPMLSRLKVYLATFRMDLSLIRDYWTLGGLLKDEGTMKGLEMVETLAKTLKLGDEAC
ETLDAERLPFGINYAMKPGTAELAPPQIYFPLLGINDGFIADALVEFFQYMGWEDQASRYKDE
LKAKFPNVDISQTKNVHRWLGVAYSETKGPSMNIYYDVVAGNVARV
>FgaPT2 (Aspergillus fumigatus tryptophan prenyltransferase,
AAX08549.1)
SEQ ID NO: 14
MKAANASSAEAYRVLSRAFRFDNEDQKLWWHSTAPMFAKMLETANYTTPCQYQYLITYKECVI
PSLGCYPTNSAPRWLSILTRYGTPFELSLNCSNSIVRYTFEPINQHTGTDKDPFNTHAIWESL
QHLLPLEKSIDLEWFRHFKHDLTLNSEESAFLAHNDRLVGGTIRTQNKLALDLKDGRFALKTY
IYPALKAVVTGKTIHELVFGSVRRLAVREPRILPPLNMLEEYIRSRGSKSTASPRLVSCDLTS
PAKSRIKIYLLEQMVSLEAMEDLWTLGGRRRDASTLEGLSLVRELWDLIQLSPGLKSYPAPYL
PLGVIPDERLPLMANFTLHQNDPVPEPQVYFTTFGMNDMAVADALTTFFERRGWSEMARTYET
TLKSYYPHADHDKLNYLHAYISFSYRDRTPYLSVYLQSFETGDWAVANLSESKVKCQDAACQP
TALPPDLSKTGVYYSGLH
>FtmPT1 (Aspergillus fumigatus Af293 brevianamide F
prenyltransferase 1, AAX56314.1)
SEQ ID NO: 15
MPPAPPDQKPCHQLQPAPYRALSESILFGSVDEERWWHSTAPILSRLLISSNYDVDVQYKYLS
LYRHLVLPALGPYPQRDPETGIIATQWRSGMVLTGLPIEFSNNVARALIRIGVDPVTADSGTA
QDPFNTTRPKVYLETAARLLPGVDLTRFYEFETELVITKAEEAVLQANPDLFRSPWKSQILTA
MDLQKSGTVLVKAYFYPQPKSAVTGRSTEDLLVNAIRKVDREGRFETQLANLQRYIERRRRGL
HVPGVTADKPPATAADKAFDACSFFPHFLSTDLVEPGKSRVKFYASERHVNLQMVEDIWTFGG
LRRDPDALRGLELLRHFWADIQMREGYYTMPRGFCELGKSSAGFEAPMMFHFHLDGSQSPFPD
PQMYVCVFGMNSRKLVEGLTTYRRVGWEEMASHYQGNFLANYPDEDFEKAAHLCAYVSFAYKN
GGAYVTLYNHSFNPVGDVSFPN
>AnaPT (Aspergillus fischeri NRRL 181 indole diterpene
prenyltransferase, A1DN10.1)
SEQ ID NO: 16
MSPLSMQTDSVQGTAENKSLETNGTSNDQQLPWKVLGKSLGLPTIEQEQYWLNTAPYFNNLLI
QCGYDVHQQYQYLAFYHRHVLPVLGPFIRSSAEANYISGFSAEGYPMELSVNYQASKATVRLG
CEPVGEFAGTSQDPMNQFMTREVLGRLSRLDPTFDLRLFDYFDSQFSLTTSEANLAASKIIKQ
RRQSKVIAFDLKDGAIIPKAYFFLKGKSLASGIPVQDVAFNAIESIAPKQIESPLRVLRTFVT
KLFSKPTVTSDVFILAVDCIVPEKSRIKLYVADSQLSLATLREFWTLGGSVTDSATMKGIEIA
EELWRILQYDDAVCSHSNMDQLPLVVNYELSSGSATPKPQLYLPLHGRNDEAMANALTKFWDY
LGWKGLAAQYKKDLYANNPCRNLAETTTVQRWVAFSYTESGGAYLTVYFHAVGGMKGNL
>XhAPT (Xylona heveae TC161 aromatic prenyltransferase,
XP_018190780.1)
SEQ ID NO: 17
MAPSMTANYPYSQISEFSKTIATSSDLDPNFGGGVSFKPSSCGGITTARKPWQILQDALGFRN
EDEHFWWETTASVLGCLLEKAGYDVHLQYQYLSLYYRYVLPSYGPRPLQPGVPHWKSFMCDDF
SPFEPSWNWDGSKSIIRFSFEPINRASGTSADPFNQIKPREVLAEISDISAGLDTQWYDHFAR
EFFLPSETASIIRSRLPEGEHMSQSFLAWDLNGGEASTKAYFFPILRSLETGRSTRDIVVDAI
TKLDSEKTSLRPSLTVLEDYMSSLPTEWQAKYEMIAIDCTDPSKSRIKIYVRMPSMAFNKVRD
MYCLGGRLHGPNVDAAMKILDDLWPRVLYIPEGTGPDDELPSNTHRTAGAIFNFELKPGNPLP
DPKLYLPVRHYAKSDLDIARGLQSFFRLQGWDEMADSYVEDLKNIFPTHDLANTAGSHTYLSY
SYKKKTGAAVTMYYNPRIYECPPVVDEVF
>PpPPT (Penicillium polonicum putative prenyltransferase,
OQD60174.1)
SEQ ID NO: 18
MTYSTATPKDSTPVSLLSLYLTFRSKDDKLWWDNTAPVIGGFLAAAHYKVASQFEFLLFYHKY
ILPSLGHYPSPENEGDRWKSFLYRRGEPLELSFNYQKDSNCTVRLALEPVGPNAGTKDDPLNE
FEAKILVEKIAQLDSNIDLQWVDFLDKEILLHNDELSQIKNTELEGSAHMSQRLVGVDFMSGG
MKIKPYFVPWLKSLVTGVPTLQLMFQAIRKLDSVGSFSNGLSEVEAYLASTDQLLWSEENYLS
FDCVDPGKSRIKLYVAEKVTCFNRIQSHWTLGGQLRSQANQEGLLLLKKLWNLLGYPGDPAQQ
TDRYLPFNFNWELRPSNPIPLPKVYFALGNEPDSLVSKALIGLFTELGWSDQIHAHKRSVEFA
FPDCNLEETTHVLTWITVTYEEEKGAYITTYCNAIGGGHKLQFR
>AtAPT (Aspergillus taichungensis aromatic prenyltransferase,
PLN85696.1)
SEQ ID NO: 19
MLLSRTTSSQNPFHLLLSGTPRLPKMRPEQEPSIQAPSKKVPLPIADGDARPWQVLSLLLPFH
NPDQKLWWDKVGPLIEIYLNCSGYNVGAQYRYLLMLHSIILPVLGPFPNSTRTHTSWPYFMNN
GDPCDLSINYQGGSAPCVRLGIEPIGPMAGTNQDPMNEYAGRRLLEDLSRIQPGIDFQLFDHF
RDTLTLSNYKARLCWHAVQEHGIKAQGHVALDLHEHSFKVKAYSIPLLRSLTSGVHYVRMMID
SIKMISRDQAITIGLSKVDEYLAATKHLLVDSRSCFSFDCADLQHSRYKIYVGANVKSLGEAY
DFWTLGGRLKGEAIDRGFQLMETIWKTMYARSLPDRKPREYIPFIWNMEVSPTDSDPIPKAYF
LVLNDYDILVSEVINCLFGELGWTEHAMTHQIIQKMAYPNHDFGSSTEIYSWISLAYKSQKGP
YITIYSNPAASL
>TgFT (Trypanosoma grayi farnesyltransferase, XP_009314693.1)
SEQ ID NO: 20
MQLREELRDAVCVFYLVLRALDTVEDDMSLAVDLKLRELPVFHEHLRDPSWRMCGVGAGRERE
LLERFPHVTRVYARLGKAYQDVITDICARMASGMCEFLTRRVESRADYDLYCHYVAGLVGHGL
TRLYVSGGFEDPNLADDLTNANHMGLFILQKTNIIRDFYEDICESPPRIFWPREIWAQTDDLH
AFKEEAHEAKALECLNAMVADALVHVPHVIEYMAALRDPSVFAFCAIPQLMAMATLALVFNNR
NVFHSKVKLTRGSTCSIILYSTQLQSAMQTMRTQAQNLLARTGPDDVCYDKIAELVGEAVRAV
DAHLQPETDGVARSMLTRYPALGGRLLYTLIDNVVGYLGK
>CoFT (Cutaneotrichosporon oleaginosum farnesyltransferase,
KLT41078.1)
SEQ ID NO: 21
MATLYPSIQSLQKFPYPGDGVVSSTLTDQHDTEGLIADVLDEQPPAHVPRLGLQNATTTLDSV
NHLKFIQGAMMSLPSGFVGLDASRPWLVFWTVHSLDLLGVLLPQNIRDRAVSTILHFLHPTGG
FCGGAANTHMPHLLPTYASVVSLAIVGNAGKGGGWERLVDARQDIYNFFMRCKRPDGGFVVGD
NCEVDVRGTYCLLVVATLLDIITPELLHNVDKAIAAGQTFEGGFACSSFTFKDGNRVAMSEAH
GGYTSCSVFSHFLLSSVQPPRRLESLPESFPVPIDVDSVVRWSAMMQGEAADGGGFRGRSNKL
VDGCYSWWVGGTFPVLEELRRREAEVKTSPNGPTATKIVAVDDDGEDEWADEASMHALFNRGM
CDSEVRLMAVALQEYTLLVAQSVTRGGLRDKPGKGPDLYHTCNNLSGLSVAQHRLTHTPEEVQ
KQREAFKADRGLPAVKPTTPGGGWKSEEERQAARREVWANVRAWVEDESDTLVVGGQMSQVNT
TVPPFNMLEVRLQPFIDYFYCQ
>SrFT (Sapingoeca rosetta farnesyltranferase, EGD76967.1)
SEQ ID NO: 22
MGYDGLVKLDPEQHLPYVTGGLGTLPSGFETLDASRPWLVYWSLNALVILGGTISPELKRRVI
NTLRMCQAETGGFGGGVGQVAHAAPTYAAVNALAIIGTEEAWSIINREKLASWLSSLIEDDGS
MHMHDDGEIDVRAVYCGASAARLCGLDVDTIFAKCPQWVARCQTYEGGFAAIPGLEAHGGYTF
CGFAAMSILCSTHLIDIPRLTEWLANRQMPMSGGFQGRPNKLVDGCYSFWVGGCFPILADLLE
AQGLPGDVVNAEALIDYVVCVCQCPSGFRDKPGKRQDYYHTSYCLSGLASMKRFAPNHPILSQ
LNATHPIHNVPPANAERMIQAMSSQTTTRH

What is claimed is:

1. A microbial cell producing 3-(4-farnesyloxyphenyl)propionic acid (FOPPA), or a derivative thereof, comprising:
an enzyme pathway for the synthesis of a first substrate that is selected from farnesyl pyrophosphate, farnesyl-phosphate, or farnesol;
an enzyme pathway for the synthesis of a second substrate that is selected from phloretate or an analog thereof, and
a transferase enzyme forming FOPPA, or a derivative thereof, from the first substrate and the second substrate.

2. The microbial cell of claim 1, wherein the analog of phloretate is selected from cinnamic acid, hydrocinnamic acid, and p-coumaric acid.

3. The microbial cell of claims 1 or 2, wherein the enzyme pathway for the synthesis of the first substrate comprises one or more farnesyl diphosphate synthases (FPPS).

4. The microbial cell of claim 3, wherein the FPPS enzyme is a Saccharomyces cerevisiae farnesyl pyrophosphate synthase (ScFPPS) having the amino acid sequence of SEQ ID NO: 1, E. coli ispA, or a variant thereof.

5. The microbial cell of any one of claims 1 to 4, wherein the enzyme pathway for the synthesis of the first substrate comprises one or more overexpressed enzymes of the methylerythritol phosphate (MEP) pathway or mevalonic acid (MVA) pathway.

6. The microbial cell of claim 5, wherein the enzyme pathway for the synthesis of the second substrate comprises: tyrosine ammonia lyase (TAL) and phenolic acid reductase (PAR).

7. The microbial cell of claim 6, wherein the enzyme pathway further comprises phenylalanine ammonia lyase (PAL) and cinnamate-4-hydroxylase (C4H).

8. The microbial cell of claim 5, wherein the enzyme pathway for the synthesis of the second substrate comprises: tyrosine ammonia lyase (TAL), phenylalanine ammonia lyase (PAL), cinnamate-4-hydroxylase (C4H), 4-coumarate-CoA ligase (4CL), hydroxycinnamoyl-CoA double bond reductase (HCDBR), chalcone synthase (CHS), and phloretin hydrolase (PH).

9. The microbial cell of claim 6 or 7, wherein the PAR comprises an amino acid sequence of wild type PAR enzyme from Clostridium spp. or Lactobacillus spp., or a derivative thereof.

10. The microbial cell of claim 6 or 7, wherein the TAL comprises an amino acid sequence of a wild type TAL enzyme from Rhodobacter spp., Rhodotorula spp., Herpatosiphon spp., Flavobacterium spp., or Saccharothrix spp., Amaranthus spp., Amborella spp., Aquilegia spp., Arabidopsis spp., Azadirachta spp., Bambusa spp., Beta spp., Cannabis spp., Capsicum spp., Carica spp., Catharanthus spp., Cistanche spp., Citrus spp., Cucumis spp., Elaeis spp., Eucalyptus spp., Glycine spp., Gossypium spp., Helianthus spp., Kalanchoë spp., Linum spp., Malus spp., Manihot spp., Mimulus spp., Musa spp., Nelumbo spp., Nicotiana spp., Orva spp., Petroselinum spp., Phalaenopsis spp., Phyllostacys spp., Physcomitrella spp., Pisum spp., Pinus spp., Populus spp., Selaginella spp., Spirodela spp., Triticum spp., Utricularia spp., Vigna spp., Vitis spp., or Zea spp., or a derivative thereof.

11. The microbial cell of claim 7, wherein the PAL comprises an amino acid sequence of a wild type PAL enzyme from Brevibacillus spp., Streptomyces spp., Dictyostelium spp., Photorhabdus spp., Amaranthus spp., Amborella spp., Aquilegia spp., Arabidopsis spp., Azadirachta spp., Bambusa spp., Beta spp., Cannabis spp., Capsicum spp., Carica spp., Catharanthus spp., Cistanche spp., Citrus spp., Cucumis spp., Elaeis spp., Eucalyptus spp., Glycine spp., Gossypium spp., Helianthus spp., Kalanchoë spp., Linum spp., Malus spp., Manihot spp., Mimulus spp., Musa spp., Nelumbo spp., Nicotiana spp., Oryza spp., Petroselinum spp., Phalaenopsis spp., Phyllostacys spp., Phycomitrella spp., Pisum spp., Pinus spp., Populus spp., Selaginella spp., Spirodela spp., Triticum spp., Utricularia spp., Vigna spp., Vitis spp., or Zea spp., or a derivative thereof.

12. The microbial cell of claim 7, wherein the C4H comprises an amino acid sequence of a wild type C4H enzyme from Amaranthus spp., Amborella spp., Aquilegia spp., Arabidopsis spp., Azadirachta spp., Bambusa spp., Beta spp., Cannabis spp., Capsicum spp., Carica spp., Catharanthus spp., Cistanche spp., Citrus spp., Cucumis spp., Elaeis spp., Eucalyptus spp., Glycine spp., Gossypium spp., Helianthus spp., Kalanchoë spp., Linum spp., Malus spp., Manihot spp., Mimulus spp., Musa spp., Nelumbo spp., Nicotiana spp., Oryza spp., Petroselinum spp., Phalaenopsis spp., Phyllostacys spp., Physcomitrella spp., Pisum spp., Pinus spp., Populus spp., Selaginella spp., Spirodela spp., Triticum spp., Utricularia spp., Vigna spp., Vitis spp., or Zea spp., or a derivative thereof.

13. The microbial cell of claim 8, wherein the 4CL enzyme comprises an amino acid sequence of a wild type 4CL enzyme from Amaranthus spp., Amborella spp., Aquilegia spp., Arabidopsis spp., Azadirachta spp., Bambusa spp., Beta spp., Camptotheca spp., Cannabis spp., Capsicum spp., Carica spp., Catharanthus spp., Cistanche spp., Citrus spp., Cucunis spp., Elaeis spp., Eucalyptus spp., Glycine spp., Gossypium spp., Helianthus spp., Kalanchoë spp., Linum spp., Malus spp., Manihot spp., Mimulus spp., Musa spp., Nelumbo spp., Nicotiana spp., Orva spp., Petroselinum spp., Phalaenopsis spp., Phyllostacys spp., Physcomitrella spp., Pisum spp., Pinus spp., Populus spp., Selaginella spp., Sesamum spp., Spirodela spp., Triticum spp., Stevia spp., Thapsia spp., Utricularia spp., Vigna spp., Vitis spp., or Zea spp., or a derivative thereof.

14. The microbial cell of claim 8, wherein the HCDBR enzyme comprises an amino acids sequence of a wild type HCDBR enzyme from Amaranthus spp., Amborella spp., Aquilegia spp., Arabidopsis spp., Azadirachta spp., Bambusa spp., Beta spp., Camptotheca spp., Cannabis spp., Capsicum spp., Carica spp., Catharanthus spp., Cistanche spp., Citrus spp., Cucumis spp., Elaeis spp., Eucalyptus spp., Glycine spp., Gossypium spp., Helianthus spp., Kalanchoë spp., Linum spp., Malus spp., Manihot spp., Mimulus spp., Musa spp., Nelumbo spp., Nicotiana spp., Oryza spp., Petroselinum spp., Phalaenopsis spp., Phyllostacys spp., Physcomitrella spp., Pisum spp., Pinus spp., Populus spp., Selaginella spp., Sesamum spp., Spirodela spp., Triticum spp., Stevia spp., Thapsia spp., Utricularia spp., Vigna spp., Vitis spp., or Zea spp., or a derivative thereof.

15. The microbial cell of claim 8, wherein the CHS enzyme comprises an amino acid sequence of a wild type CHS enzyme from Amaranthus spp., Amborella spp., Aquilegia spp., Arabidopsis spp., Azadirachta spp., Bambusa spp., Beta spp., Camptotheca spp., Cannabis spp., Capsicum spp., Carica spp., Catharanthus spp., Cistanche spp., Citrus spp., Cucunis spp., Elaeis spp., Eucalyptus spp., Glycine spp., Gossypium spp., Helianthus spp., Kalanchoë spp., Linum spp., Malus spp., Manihot spp., Mimulus spp., Musa spp., Nelumbo spp., Nicotiana spp., Oryza spp., Petroselinum spp., Phalaenopsis spp., Phyllostacys spp., Physcomitrella spp., Pisum spp., Pinus spp., Populus spp., Selaginella spp., Sesamum spp., Spirodela spp., Triticum spp., Stevia spp., Thapsia spp., Utricularia spp., Vigna spp., Vitis spp., or Zea spp., or a derivative thereof.

16. The microbial cell of claim 8, wherein the PH enzyme comprises a wild type PH enzyme from Acidaminococcus spp., Anaerovibrio spp., Aspergillus spp., eButyricicoccus spp., Canis spp., Clostridium spp., Dialister spp., Erwinia spp., Eubacterium spp., Flavonfractor spp., Flavonfractor sp. An112, Homo spp., Lachnospira spp., Megasphaera spp., Mus spp., Oribacterium spp., Oryctolagus spp., Pantoca spp., Parasporobacterium spp., Propionispira spp., Ratus spp., Roseburia spp., Selenomonas spp., or Sharpea spp., or a derivative thereof.

17. The microbial cell of any one of claims 6 to 16, wherein the enzyme pathway for the synthesis of the second substrate comprises one or more cytochrome P450 reductases (CPR).

18. The microbial cell of claim 6 or 7, wherein the enzyme pathway for the synthesis of the second substrate comprises an enzyme involved in the conversion of p-coumaric acid to phloretate in Lactobacillus plantarum.

19. The microbial cell of claim 6 or 7, wherein the enzyme pathway for the synthesis of the second substrate comprises an enzyme involved in the production of phloretate from tyrosine by Clostridium orbiscindens.

20. The microbial cell of any one of claims 1 to 19, wherein the transferase enzyme comprises an amino acid sequence of Aspergillus terreus aromatic Prenyl Transferase (AtaPT) having an accession number selected from KP893683, EAU39348, EAU39467, EAU36097, EAU36020, EAU31601, EAU29429, EAU29303, or a variant thereof.

21. The microbial cell of claim 20, wherein the transferase enzyme comprises an amino acid sequence having at least about 70% amino acid sequence identity with any one of KP893683, EAU39348, EAU39467, EAU36097, EAU36020, EAU31601, EAU29429, and EAU29303.

22. The microbial cell of any one of claims 1 to 21 wherein the transferase enzyme comprises an amino acid sequence selected from SEQ ID NOs: 2 to 22, or a variant thereof.

23. The microbial cell of claim 22, wherein the transferase enzyme comprises an amino acid sequence having at least about 70% amino acid sequence identity to any one of SEQ ID NOs: 2 to 22.

24. The microbial cell of claim 23, wherein the transferase enzyme comprises the amino acid sequence of SEQ ID NO: 2 with one or more of the following modifications: deletion of amino acids corresponding to amino acids 1-10 of SEQ ID NO: 2 and a substitution at one or more positions corresponding to H88, E91, S177, or W397 of SEQ ID NO: 2.

25. The microbial cell of claim 24, wherein the transferase comprises a substitution selected from H88A, E91A, E91Q, E91D, S177A, and W397A.

26. The microbial cell of claim 23, wherein the transferase enzyme comprises the amino acid sequence of SEQ ID NO: 3 with one or more substitutions at positions corresponding to W97, E123, F170, A173, and F189 of SEQ ID NO: 3.

27. The microbial cell of claim 26, wherein the transferase enzyme comprises a substitution selected from W97Y and A173M.

28. The microbial cell of claim 26, wherein the transferase enzyme comprises the amino acid sequence of SEQ ID NO: 4 with one or more substitutions at positions corresponding to Y80, W157, and M159 of SEQ ID NO: 4.

29. The microbial cell of claim 28, wherein the transferase enzyme comprises a substitution selected from Y80W and M159A.

30. The microbial cell of any one of claims 1 to 29, wherein at least one enzyme is a circular permutant.

31. The microbial cell of any one of claims 1 to 30, wherein the cell produces a derivative of FOPPA selected from 3-(4-farnesyloxyphenyl)-propionic acid methyl ester, 4-farnesyloxycinnamic acid methyl ester, and 4-farnesyloxycinnamic acid.

32. The microbial cell of any one of claims 1 to 31, wherein the microbial cell is prokaryotic or eukaryotic.

33. The microbial cell of claim 32, wherein the microbial cell is a bacteria cell, which is optionally E. coli.

34. The microbial cell of claim 32, wherein the microbial cell is a yeast cell.

35. A method for making FOPPA, or a derivative thereof, comprising:
culturing the microbial cell of any one of claims 1 to 34, and recovering FOPPA, or a derivative thereof, from the cells or from the culture.

36. A method for making FOPPA, or a derivative thereof, comprising:
contacting a first substrate and a second substrate with a prenyltransferase to make FOPPA, or a derivative thereof,
wherein the first substrate is selected from farnesyl pyrophosphate, farnesyl-phosphate, or farnesol;
wherein the second substrate is selected from phloretate or a precursor or analog thereof, and
wherein the prenyltransferase comprises an amino acid sequence selected from Aspergillus terreus aromatic Prenyl Transferase (AtaPT) having an accession number selected from KP893683, EAU39348, EAU39467, EAU36097, EAU36020, EAU31601, EAU29429, and EAU29303, or a variant thereof, and a transferase enzyme comprising an amino acid sequence selected from SEQ ID NOs: 2 to 22 or a variant thereof.

37. The method of claim 36, wherein the precursor or analog of phloretate is selected from cinnamic acid, hydrocinnamic acid, and p-coumaric acid.

38. The method of claim 36 or 27, wherein the prenyltransferase enzyme comprises an amino acid sequence selected from accession numbers KP893683, EAU39348, EAU39467, EAU36097, EAU36020, EAU31601, EAU29429, and EAU29303, or a derivative thereof.

39. The method of claim 36 or 37, wherein the prenyltransferase enzyme comprises an amino acid sequence of any one of SEQ ID NOs: 2 to 22, or a derivative thereof.

40. The method of claim 39, wherein the prenyltransferase enzyme comprises the amino acid sequence of SEQ ID NO: 2 having one or more of the following modifications: deletion of amino acids corresponding to amino acids 1-10 of SEQ ID NO: 2 and a substitution at a position corresponding to H88, E91, S177, or W397 of SEQ ID NO: 2.

41. The method of claim 40, wherein the prenyltransferase enzyme comprises a substitution selected from H88A, E91A, E91Q, E91D, S177A, and W397A.

42. The method of claim 39, wherein the prenyltransferase enzyme comprises the amino acid sequence of SEQ ID NO: 3 with one or more substitutions at positions corresponding to W97, E123, F170, A173, and F189 of SEQ ID NO: 3.

43. The method of claim 42, wherein the prenyltransferase enzyme comprises a substitution selected from W97Y and A173M.

44. The method of claim 39, wherein the prenyltransferase enzyme comprises the amino acid sequence of SEQ ID NO: 4 with one or more substitutions at positions corresponding to Y80, W157, and M159 of SEQ ID NO: 3.

45. The method of claim 44, wherein the prenyltransferase enzyme comprises a substitution selected from Y80W and M159A.

46. The method of any one of claims 36 to 45, wherein at least one enzyme is a circular permutant.

47. The method of any one of claims 36 to 46, wherein the prenyltransferase is expressed in a microbe and contacted with the first substrate and the second substrate in the form of whole cells expressing the prenyltransferase, cellular extract, or in purified form.

48. The method of any one of claims 36 to 46, wherein the prenyltransferase is expressed in a microbe, wherein the microbe overexpresses an enzyme in the pathway for the synthesis of the first substrate.

49. The method of claim 47, wherein the phloretate or an analog thereof is fed to the culture or reaction.

50. The method of any one of claims 36 to 49, wherein the phloretate, or a derivative thereof, is prepared from a phloretate precursor selected from L-phenylalanine, cinnamic acid, tyrosine, p-coumaric acid, p-coumaroyl-CoA, p-dihydrocoumaroyl-CoA, phloretin, p-hydroxyphenylpyruvic acid, and p-hydroxyphenyllactic acid by a reaction with one or more enzymes for producing the phloretate or a derivative thereof.

51. The method of claim 47, wherein the cellular extract is an extract of a microbe overexpressing the prenyltransferase, and optionally overexpressing an enzyme to increase production of farnesyl pyrophosphate, farnesyl-phosphate, or farnesol.

52. The method of claim 36, wherein the farnesyl pyrophosphate, farnesyl-phosphate, and/or farnesol are provided in a cell free system comprising the prenyltransferase and at least one microbial cell engineered to produce the phloretate, or a derivative thereof.

53. The method of claim 52, wherein the microbial cell expresses an enzyme pathway for the synthesis of phloretate, which comprises: tyrosine ammonia lyase (TAL) and phenolic acid reductase (PAR).

54. The method of claim 53, wherein the enzyme pathway further comprises phenylalanine ammonia lyase (PAL) and cinnamate-4-hydroxylase (C4H).

55. The method of claim 52, wherein the enzyme pathway for the synthesis of phloretate comprises: tyrosine ammonia lyase (TAL), phenylalanine ammonia lyase (PAL), cinnamate-4-hydroxylase (C4H), 4-coumarate-CoA ligase (4CL), hydroxycinnamoyl-CoA double bond reductase (HCDBR), chalcone synthase (CHS), and phloretin hydrolase (PH).

56. The method of claim 53 or 54, wherein the PAR comprises an amino acid sequence of wild type PAR enzyme from Clostridium spp. or Lactobacillus spp., or a derivative thereof.

57. The method of claim 53 or 54, wherein the TAL comprises an amino acid sequence of a wild type TAL enzyme from Rhodobacter spp., Rhodotorula spp., Herpatosiphon spp., Flavobacterium spp., or Saccharothrix spp., Amaranthus spp., Amborella spp., Aquilegia spp., Arabidopsis spp., Azadirachta spp., Bambusa spp., Beta spp., Cannabis spp., Capsicum spp., Carica spp., Catharanthus spp., Cistanche spp., Citrus spp., Cucumis spp., Elaeis spp., Eucalyptus spp., Glycine spp., Gossypium spp., Helianthus spp., Kalanchoë spp., Linum spp., Malus spp., Manihot spp., Mimulus spp., Musa spp., Nelumbo spp., Nicotiana spp., Oryza spp., Petroselinum spp., Phalaenopsis spp., Phyllostacys spp., Physcomitrella spp., Pisum spp., Pinus spp., Populus spp., Selaginella spp., Spirodela spp., Triticum spp., Utricularia spp., Vigna spp., Vitis spp., or Zea spp., or a derivative thereof.

58. The method of claim 55, wherein the PAL comprises an amino acid sequence of a wild type PAL enzyme from Brevibacillus spp., Streptomyces spp., Dictyostelium spp., Photorhabdus spp., Amaranthus spp., Amborella spp., Aquilegia spp., Arabidopsis spp., Azadirachta spp., Bambusa spp., Beta spp., Cannabis spp., Capsicum spp., Carica spp., Catharanthus spp., Cistanche spp., Citrus spp., Cucumis spp., Elaeis spp., Eucalyptus spp., Glycine spp., Gossypium spp., Helianthus spp., Kalanchoë spp., Linum spp., Malus spp., Manihot spp., Mimulus spp., Musa spp., Nelumbo spp., Nicotiana spp., Oryza spp., Petroselinum spp., Phalaenopsis spp., Phyllostacys spp., Physcomitrella spp., Pisum spp., Pinus spp., Populus spp., Selaginella spp., Spirodela spp., Triticum spp., Utricularia spp., Vigna spp., Vitis spp., or Zea spp., or a derivative thereof.

59. The method of claim 55, wherein the C4H comprises an amino acid sequence of a wild type C4H enzyme from Amaranthus spp., Amborella spp., Aquilegia spp., Arabidopsis spp., Azadirachta spp., Bambusa spp., Beta spp., Cannabis spp., Capsicum spp., Carica spp., Catharanthus spp., Cistanche spp., Citrus spp., Cucumis spp., Elaeis spp., Eucalyptus spp., Glycine spp., Gossypium spp., Helianthus spp., Kalanchoë spp., Linum spp., Malus spp., Manihot spp., Mimulus spp., Musa spp., Nelumbo spp., Nicotiana spp., Oryza spp., Petroselinum spp., Phalaenopsis spp., Phyllostacys spp., Physcomitrella spp., Pisum spp., Pinus spp., Populus spp., Selaginella spp., Spirodela spp., Triticum spp., Utricularia spp., Vigna spp., Vitis spp., or Zea spp., or a derivative thereof.

60. The method of claim 55, wherein the 4CL enzyme comprises an amino acid sequence of a wild type 4CL enzyme from Amaranthus spp., Amborella spp., Aquilegia spp., Arabidopsis spp., Azadirachta spp., Bambusa spp., Beta spp., Camptotheca spp., Cannabis spp., Capsicum spp., Carica spp., Catharanthus spp., Cistanche spp., Citrus spp., Cucumis spp., Elaeis spp., Eucalyptus spp., Glycine spp., Gossypium spp., Helianthus spp., Kalanchoë spp., Linum spp., Malus spp., Manihot spp., Mimulus spp., Musa spp., Nelumbo spp., Nicotiana spp., Oryza spp., Petroselinum spp., Phalaenopsis spp., Phyllostacys spp., Physcomitrella spp., Pisum spp., Pinus spp., Populus spp., Selaginella spp., Sesamum spp., Spirodela spp., Triticum spp., Stevia spp., Thapsia spp., Utricularia spp., Vigna spp., Vitis spp., or Zea spp., or a derivative thereof.

61. The method of claim 55, wherein the HCDBR enzyme comprises an amino acids sequence of a wild type HCDBR enzyme from Amaranthus spp., Amborella spp., Aquilegia spp., Arabidopsis spp., Azadirachta spp., Bambusa spp., Beta spp., Camptotheca spp., Cannabis spp., Capsicum spp., Carica spp., Catharanthus spp., Cistanche spp., Citrus spp., Cucumis spp., Elaeis spp., Eucalyptus spp., Glycine spp., Gossypium spp., Helianthus spp., Kalanchoë spp., Linum spp., Malus spp., Manihot spp., Mimulus spp., Musa spp., Nelumbo spp., Nicotiana spp., Oryza spp., Petroselinum spp., Phalaenopsis spp., Phyllostacys spp., Physcomitrella spp., Pisum spp., Pinus spp., Populus spp., Selaginella spp., Sesamum spp., Spirodela spp., Triticum spp., Stevia spp., Thapsia spp., Utricularia spp., Vigna spp., Vitis spp., or Zea spp., or a derivative thereof.

62. The method of claim 55, wherein the CHS enzyme comprises an amino acid sequence of a wild type CHS enzyme from Amaranthus spp., Amborella spp., Aquilegia spp., Arabidopsis spp., Azadirachta spp., Bambusa spp., Beta spp., Camptotheca spp., Cannabis spp., Capsicum spp., Carica spp., Catharanthus spp., Cistanche spp., Citrus spp., Cucumis spp., Elaeis spp., Eucalyptus spp., Glycine spp., Gossypium spp., Helianthus spp., Kalanchoë spp., Linum spp., Malus spp., Manihot spp., Mimulus spp., Musa spp., Nelumbo spp., Nicotiana spp., Oryza spp., Petroselinum spp., Phalaenopsis spp., Phyllostacys spp., Physcomitrella spp., Pisum spp., Pinus spp., Populus spp., Selaginella spp., Sesamum spp., Spirodela spp., Triticum spp., Stevia spp., Thapsia spp., Utricularia spp., Vigna spp., Vitis spp., or Zea spp., or a derivative thereof.

63. The method of claim 55, wherein the PH enzyme comprises a wild type PH enzyme from Acidaminococcus spp., Anaerovibrio spp., Aspergillus spp., eButyricicoccus spp., Canis spp., Clostridium spp., Dialister spp., Erwinia spp., Eubacterium spp., Flavonifractor spp., Flavonifractor sp. An112, Homo spp., Lachnospira spp., Megasphaera spp., Mus spp., Oribacterium spp., Oryctolagus spp., Pantoea spp., Parasporobacterium spp., Propionispira spp., Ratus spp., Roseburia spp., Selenomonas spp., or Sharpea spp., or a derivative thereof.

64. The method of any one of claims 52 to 63, wherein the enzyme pathway for the synthesis of phloretate or an analog thereof comprises one or more cytochrome P450 reductases (CPR).

65. The method of claim 53 or 54, wherein the enzyme pathway for the synthesis of the phloretate or precursor or analog thereof comprises an enzyme involved in the conversion of p-coumaric acid to phloretate in Lactobacillus plantarum.

66. The method of claim 53 or 54, wherein the enzyme pathway for the synthesis of the phloretate or precursor or analog thereof comprises an enzyme involved in the production of phloretate from tyrosine by Clostridium orbiscindens.

67. The method of any one of claims 36 to 46, wherein the farnesyl pyrophosphate, farnesyl-phosphate, or farnesol are produced in a microbial cell.

68. The method of any one of claims 36 to 67, further comprising harvesting the FOPPA from the cell culture or reaction.

69. A method for making a product comprising FOPPA, or a derivative thereof, comprising producing FOPPA, or a derivative thereof, according to the method of any one of claims 35 to 69, and incorporating the FOPPA, or a derivative thereof, into the product.

70. The method of claim 69, wherein the product is a skin-lightening composition.

71. The method of claim 69, wherein the product is an anti-seborrheic composition.

72. The method of claim 69, wherein the product is a composition for use in any of the applications selected from antioxidants, antibacterials, anthelmintic, anti-inflammatories, cancer chemopreventatives, food additives, and fragrance components in pharmaceuticals, nutraceuticals, foods and cosmetics.


SEQUENCE LISTING (22 sequences)

SEQ ID NO: 1 - protein, 352 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 2 - protein, 424 aa, Aspergillus terreus
MLPPSDSKDPRPWQILSQALGFPNYDQELWWQNTAETLNRVLEQCDYSVHLQYKYLAFYHKYILPSLGPFRRPGVEPEYISGLSHGGHPLEISVKIDKSKTICRLGLQAIGPLAGTARDPLNSFGDRELLKNLATLLPHVDLRLFDHFNAQVGLDRAQCAVATTKLIKESHNIVCTSLDLKDGEVIPKVYFSTIPKGLVTETPLFDLTFAAIEQMEVYHKDAPLRTALSSLKDFLRPRVPTDASITPPLTGLIGVDCIDPMLSRLKVYLATFRMDLSLIRDYWTLGGLLTDAGTMKGLEMVETLAKTLKLGDEACETLDAERLPFGINYAMKPGTAELAPPQIYFPLLGINDGFIADALVEFFQYMGWEDQANRYKDELKAKFPNVDISQTKNVHRWLGVAYSETKGPSMNIYYDVVAGNVARV

SEQ ID NO: 3 - protein, 391 aa, Streptomyces blastmyceticus
MESAGPGTGPQPPRTSGDFTPDTGVIAEMTGRPMRFDSDRYRPTDTYAEVACDKVCRAYEGLGADGGDRESLLAFLRDLTDPWGELPVGTPPEDACWVSIDGMPLETSVAWAGRKAGVRLSLESPRGPAKRRMEDGMALTRRLAGRPGVSVDPCLRVEDLFTDDDPQGYFTIAHAVAWTPGGHPRYKIFLNPAVRGREQAAARTEEAMIRLGLEQPWRALTEHLGGAYGPEHEPAALAMDLVPGDDFRVQVYLAHSGVSAEAIDAKSAVAADHVPGSFARALRGINGADDTPEWKRKPPVTAFSFGPGRAVPGATLYVPMIPVHGSDAAARDRVAAFLRSEGMDAVGYEAVLDAISDRSLPESHTQNFISYRGGDSPRFSVYLAPGVYREA

SEQ ID NO: 4 - protein, 374 aa, Marinactinospora thermotolerans
MAGDPFVDNGTVSSQRPLRAVPGRYPPGATHLDAAVDTLVRCHAALGRAPSEAEAAVCLLRRLWGRWGNTPVERPGWRSYVAVDGSPFELSAAWNGDGPAEVRVTVEATADPPTPEGNQEAGWEYLRGLSRHPGAATARVLALEDLFRPQTPHDRCWIMHGMASRPGADPLFKVYLDPDARGAAEAPSVLDEAMDRLGVRAAWQGLRGWLDEHGGSGRIGSLALDLADTDDARVKVYVQHAGLDWADIDRQAAVARGHVPGAFSAALEEITGTEVPPHKPPVTCFAFHRGVGVPTAATLYIPMPAGVPESDARRRSAAFMRRSGLDSAAYLAFLAAATGDGEGVRALQNFVAYRPAAPGGRPRFACYVAPGLYR

SEQ ID NO: 5 - protein, 439 aa, Pestalotiopsis fici
MAISTPSNGVSHVAKPLPNLKEVNKGIETDSEDRAFWWGALSEPLASLLEANHYTKEVQLHYLRWFYQWILPALGPRPLDGKPYYGSWITHDLSPFEYSLNWKEKSSKQTIRFTIEAVTKQSGTASDPINQLGAKEFLEAVSKDVPGMDLTRFNQFLEATNVPNDCVDDAIAKHPAHFPRSRVWIAFDLEHSGNLMAKSYFLPHWRAIQSGISANTIIGDTVKECNKADGSSYDGSLNAIESYLATFTRPEEAPQMGLLSNDCVAETPGSRLKVYFRSSADTLAKAKDMYNLGGRLKGPKMDASLKGISDFWYHLFGLDSSDPASDDKVCIGNHKCIFVYEMRSSQGSEPDIDVKFHIPMWQLGKTDGQISELLASWFESHGHPDLASRYKSDLGTAFPKHNITGKSVGTHTYISITHTPKTGLYMTMYLSPKLPEFYY

SEQ ID NO: 6 - protein, 316 aa, Streptomyces sp.
MIGIDFLECLVSEGIEAEGLYSAIEESARMVDAPFSRDKVWPILSAFGGGFSDAGGVIFSLQAGKDVPEMEYSAQISAEVGDPYAHALATGVLNETDHPVSTVLAEIVSLAPTSEHYIDCGIVGGFKKIYANFPHDQQKVSRLADLPAMPRAVGANAEFFDRYGLDNVALIGVDYRNKTINLYFQAPAETAGNLDPKTVSAMLRETGMSTPSEEMVAYADRAYRIYATLGWDSPEVMRLAFAPQPRRSIDLAELPARLEPRIEQFMRATPHKYPGALINATAAKWSKKHEVLDLAAYYQVSALHLKAIQAEEGQSS

SEQ ID NO: 7 - protein, 300 aa, Streptomyces cinnamonensis
MMSGTADLAGVYAAVEESAGLLDVSCAREKVWPILAAFEDVLPTAVIAFRVATNARHEGEFDCRFTVPGSIDPYAVALDKGLTHRSGHPIETLVADVQKHCAVDSYGVDFGVVGGFKKIWVYFPGGRHESLAHLGEIPSMPPGLAATEGFFARYGLADKVDLIGVDYASKTMNVYFAASPEVVSAPTVLAMHREIGLPDPSEQMLDFCSRAFGVYTTLNWDSSKVERIAYSVKTEDPLELSARLGSKVEQFLKSVPYGIDTPKMVYAAVTAGGEEYYKLQSYYQWRTDSRLNLSYIGGRS

SEQ ID NO: 8 - protein, 307 aa, Streptomyces sp.
MPGTDDVAVDVASVYSAIEKSAGLLDVTAAREVVWPVLTAFEDVLEQAVIAFRVATNARHEGDFDVRFTVPEEVDPYAVALSRSLIAKTDHPVGSLLSDIQQLCSVDTYGVDLGVKSGFKKVWVYFPAGEHETLARLTGLTSMPGSLAGNVDFFTRYGLADKVDVIGIDYRSRTMNVYFAAPSECFERETVLAMHRDIGLPSPSEQMFKFCENSFGLYTTLNWDTMEIERISYGVKTENPMTFFARLGTKVEHFVKNVPYGVDTQKMVYAAVTSSGEEYYKLQSYYRWRSVSRLNAAYIAARDKEST

SEQ ID NO: 9 - protein, 435 aa, Aspergillus versicolor
MTAPELRAPAGHPQEPPARSSPAQALSSYHHFPTSDQERWYQETGSLCSRFLEAGQYGLHQQYQFMFFFMHHLIPALGPYPQKWRSTISRSGLPIEFSLNFQKGSHRLLRIGFEPVNFLSGSSQDPFNRIPIADLLAQLARLQLRGFDTQCFQQLLTRFQLSLDEVRQLPPDDQPLKSQGAFGFDFNPDGAILVKGYVFPYLKAKAAGVPVATLIAESVRAIDADRNQFMHAFSLINDYMQESTGYNEYTFLSCDLVEMSRQRVKIYGAHTEVTWAKIAEMWTLGGRLIEEPEIMEGLARLKQIWSLLQIGEGSRAFKGGFDYGKASATDQIPSPIIWNYEISPGSSFPVPKFYLPVHGENDLRVARSLAQFWDSLGWSEHACAYPDMLQQLYPDLDVSRTSRLQSWISYSYTAKKGVYMSVYFHSQSTYLWEED

SEQ ID NO: 10 - protein, 472 aa, Aspergillus fumigatus
MSIGAEIDSLVPAPPGLNGTAAGYPAKTQKELSNGDFDAHDGLSLAQLTPYDVLTAALPLPAPASSTGFWWRETGPVMSKLLAKANYPLYTHYKYLMLYHTHILPLLGPRPPLENSTHPSPSNAPWRSFLTDDFTPLEPSWNVNGNSEAQSTIRLGIEPIGFEAGAAADPFNQAAVTQFMHSYEATEVGATLTLFEHFRNDMFVGPETYAALRAKIPEGEHTTQSFLAFDLDAGRVTTKAYFFPILMSLKTGQSTTKVVSDSILHLALKSEVWGVQTIAAMSVMEAWIGSYGGAAKTEMISVDCVNEADSRIKIYVRMPHTSLRKVKEAYCLGGRLTDENTKEGLKLLDELWRTVFGIDDEDAELPQNSHRTAGTIFNFELRPGKWFPEPKVYLPVRHYCESDMQIASRLQTFFGRLGWHNMEKDYCKHLEDLFPHHPLSSSTGTHTFLSFSYKKQKGVYMTMYYNLRVYST

SEQ ID NO: 11 - protein, 440 aa, Aspergillus fumigatus
MDGEMTASPPDISACDTSAVDEQTGQSGQSQAPIPKDIAYHTLTKALLFPDIDQYQHWHHVAPMLAKMLVDGKYSIHQQYEYLCLFAQLVAPVLGPYPSPGRDVYRCTLGGNMTVELSQNFQRSGSTTRIAFEPVRYQASVGHDRFNRTSVNAFFSQLQLLVKSVNIELHHLLSEHLTLTAKDERNLNEEQLTKYLTNFQVKTQYVVALDLRKTGIVAKEYFFPGIKCAATGQTGSNACFGAIRAVDKDGHLDSLCQLIEAHFQQSKIDDAFLCCDLVDPAHTRFKVYIADPLVTLARAEEHWTLGGRLTDEDAAVGLEIIRGLWSELGIIQGPLEPSAMMEKGLLPIMLNYEMKAGQRLPKPKLYMPLTGIPETKIARIMTAFFQRHDMPEQAEVFMENLQAYYEGKNLEEATRYQAWLSFAYTKEKGPYLSIYYFWPE

SEQ ID NO: 12 - protein, 446 aa, Aspergillus oryzae
MSLRNDLDNGRPTKRLESWDIASMWLSDRKDEIQDWWDFSGPQLATLAHEAGYSTMTQIELLLFFRSVVLPRMGRFPDACRPRACAQSRSILTYDGSPIEYSWKWNNSANDHPEIRFCVEPVGDGLCADGIVGGKLRATDEILVQLAKRVPSTDLEWYHHFRDSFGLGHWTDGPLHEDAGTWQVRRPRMPVAFEFTPKGIVTKVYFTPPATLDDMPSFNMFADVVRPIGDKDTTALDESMEYLSRDPVGATLRPDVLAIDCISPLKSRIKLYAGTAMTTFTSAISVLTLGGRIPVTRHSIDEMWALFRMVLGLHDKFLQDEELPVQNPFQPSRAHPEDYYSGLLYYFNLAPGALLPDVKLYLPVIRYGRSDADIALGLQRFMASRHRGQYVDGFQRAMEIISQRHKSGNGHRIQTYIACSFDKDGSLSLTSYLNPGVYFSSETVDV

SEQ ID NO: 13 - protein, 424 aa, Aspergillus terreus
MLPPSDSKDPRPWQILSQALGFPNYDQELWWQNTAETLNRVLEQCDYSVHLQYKYLAFYHKYILPSLGPFRRPGVEPEYISGLSHGGHPLEISVKIDKSKTICRLGLQAIGPLAGTARDPLNSFGDRELLKNLATLLPHVDLRLFDHFNAQVGLDRAQCAVATTKLIKESHNIVCTSLDLKDGEVIPKVYFSTIPKGLVTETPLFDLTFAAIEQMEVYHKDAPLRTALSSLKDFLRPRVPTDASITPPLTGLIGVDCIDPMLSRLKVYLATFRMDLSLIRDYWTLGGLLKDEGTMKGLEMVETLAKTLKLGDEACETLDAERLPFGINYAMKPGTAELAPPQIYFPLLGINDGFIADALVEFFQYMGWEDQASRYKDELKAKFPNVDISQTKNVHRWLGVAYSETKGPSMNIYYDVVAGNVARV

SEQ ID NO: 14 - protein, 459 aa, Aspergillus fumigatus
MKAANASSAEAYRVLSRAFRFDNEDQKLWWHSTAPMFAKMLETANYTTPCQYQYLITYKECVIPSLGCYPTNSAPRWLSILTRYGTPFELSLNCSNSIVRYTFEPINQHTGTDKDPFNTHAIWESLQHLLPLEKSIDLEWFRHFKHDLTLNSEESAFLAHNDRLVGGTIRTQNKLALDLKDGRFALKTYIYPALKAVVTGKTIHELVFGSVRRLAVREPRILPPLNMLEEYIRSRGSKSTASPRLVSCDLTSPAKSRIKIYLLEQMVSLEAMEDLWTLGGRRRDASTLEGLSLVRELWDLIQLSPGLKSYPAPYLPLGVIPDERLPLMANFTLHQNDPVPEPQVYFTTFGMNDMAVADALTTFFERRGWSEMARTYETTLKSYYPHADHDKLNYLHAYISFSYRDRTPYLSVYLQSFETGDWAVANLSESKVKCQDAACQPTALPPDLSKTGVYYSGLH

SEQ ID NO: 15 - protein, 463 aa, Aspergillus fumigatus
MPPAPPDQKPCHQLQPAPYRALSESILFGSVDEERWWHSTAPILSRLLISSNYDVDVQYKYLSLYRHLVLPALGPYPQRDPETGIIATQWRSGMVLTGLPIEFSNNVARALIRIGVDPVTADSGTAQDPFNTTRPKVYLETAARLLPGVDLTRFYEFETELVITKAEEAVLQANPDLFRSPWKSQILTAMDLQKSGTVLVKAYFYPQPKSAVTGRSTEDLLVNAIRKVDREGRFETQLANLQRYIERRRRGLHVPGVTADKPPATAADKAFDACSFFPHFLSTDLVEPGKSRVKFYASERHVNLQMVEDIWTFGGLRRDPDALRGLELLRHFWADIQMREGYYTMPRGFCELGKSSAGFEAPMMFHFHLDGSQSPFPDPQMYVCVFGMNSRKLVEGLTTYRRVGWEEMASHYQGNFLANYPDEDFEKAAHLCAYVSFAYKNGGAYVTLYNHSFNPVGDVSFPN

SEQ ID NO: 16 - protein, 437 aa, Aspergillus fischeri
MSPLSMQTDSVQGTAENKSLETNGTSNDQQLPWKVLGKSLGLPTIEQEQYWLNTAPYFNNLLIQCGYDVHQQYQYLAFYHRHVLPVLGPFIRSSAEANYISGFSAEGYPMELSVNYQASKATVRLGCEPVGEFAGTSQDPMNQFMTREVLGRLSRLDPTFDLRLFDYFDSQFSLTTSEANLAASKLIKQRRQSKVIAFDLKDGAIIPKAYFFLKGKSLASGIPVQDVAFNAIESIAPKQIESPLRVLRTFVTKLFSKPTVTSDVFILAVDCIVPEKSRIKLYVADSQLSLATLREFWTLGGSVTDSATMKGLEIAEELWRILQYDDAVCSHSNMDQLPLVVNYELSSGSATPKPQLYLPLHGRNDEAMANALTKFWDYLGWKGLAAQYKKDLYANNPCRNLAETTTVQRWVAFSYTESGGAYLTVYFHAVGGMKGNL

SEQ ID NO: 17 - protein, 470 aa, Xylona heveae
MAPSMTANYPYSQISEFSKTIATSSDLDPNFGGGVSFKPSSCGGITTARKPWQILQDALGFRNEDEHFWWETTASVLGCLLEKAGYDVHLQYQYLSLYYRYVLPSYGPRPLQPGVPHWKSFMCDDFSPFEPSWNWDGSKSIIRFSFEPINRASGTSADPFNQIKPREVLAEISDISAGLDTQWYDHFAREFFLPSETASIIRSRLPEGEHMSQSFLAWDLNGGEASTKAYFFPILRSLETGRSTRDIVVDAITKLDSEKTSLRPSLTVLEDYMSSLPTEWQAKYEMIAIDCTDPSKSRIKIYVRMPSMAFNKVRDMYCLGGRLHGPNVDAAMKILDDLWPRVLYIPEGTGPDDELPSNTHRTAGAIFNFELKPGNPLPDPKLYLPVRHYAKSDLDIARGLQSFFRLQGWDEMADSYVEDLKNIFPTHDLANTAGSHTYLSYSYKKKTGAAVTMYYNPRIYECPPVVDEVF

SEQ ID NO: 18 - protein, 422 aa, Penicillium polonicum
MTYSTATPKDSTPVSLLSLYLTFRSKDDKLWWDNTAPVIGGFLAAAHYKVASQFEFLLFYHKYILPSLGHYPSPENEGDRWKSFLYRRGEPLELSFNYQKDSNCTVRLALEPVGPNAGTKDDPLNEFEAKILVEKIAQLDSNIDLQWVDFLDKEILLHNDELSQIKNTELEGSAHMSQRLVGVDFMSGGMKIKPYFVPWLKSLVTGVPTLQLMFQAIRKLDSVGSFSNGLSEVEAYLASTDQLLWSEENYLSFDCVDPGKSRIKLYVAEKVTCFNRIQSHWTLGGQLRSQANQEGLLLLKKLWNLLGYPGDPAQQTDRYLPFNFNWELRPSNPIPLPKVYFALGNEPDSLVSKALIGLFTELGWSDQIHAHKRSVEFAFPDCNLEETTHVLTWITVTYEEEKGAYITTYCNAIGGGHKLQFR

SEQ ID NO: 19 - protein, 453 aa, Aspergillus taichungensis
MLLSRTTSSQNPFHLLLSGTPRLPKMRPEQEPSIQAPSKKVPLPIADGDARPWQVLSLLLPFHNPDQKLWWDKVGPLIEIYLNCSGYNVGAQYRYLLMLHSIILPVLGPFPNSTRTHTSWPYFMNNGDPCDLSINYQGGSAPCVRLGIEPIGPMAGTNQDPMNEYAGRRLLEDLSRIQPGIDFQLFDHFRDTLTLSNYKARLCWHAVQEHGIKAQGHVALDLHEHSFKVKAYSIPLLRSLTSGVHYVRMMIDSIKMISRDQAITIGLSKVDEYLAATKHLLVDSRSCFSFDCADLQHSRYKIYVGANVKSLGEAYDFWTLGGRLKGEAIDRGFQLMETIWKTMYARSLPDRKPREYIPFIWNWEVSPTDSDPIPKAYFLVLNDYDILVSEVINCLFGELGWTEHAMTHQIIQKMAYPNHDFGSSTEIYSWISLAYSQSKGPYITIYSNPAASL

SEQ ID NO: 20 - protein, 355 aa, Trypanosoma grayi
MQLREELRDAVCVFYLVLRALDTVEDDMSLAVDLKLRELPVFHEHLRDPSWRMCGVGAGRERELLERFPHVTRVYARLGKAYQDVITDICARMASGMCEFLTRRVESRADYDLYCHYVAGLVGHGLTRLYVSGGFEDPNLADDLTNANHMGLFLQKTNIIRDFYEDICESPPRIFWPREIWAQYTDDLHAFKEEAHEAKALECLNAMVADALVHVPHVIEYMAALRDPSVFAFCAIPQLMAMATLALVFNNRNVFHSKVKLTRGSTCSIILYSTQLQSAMQTMRTQAQNLLARTGPDDVCYDKIAELVGEAVRAVDAHLQPETDGVARSMLTRYPALGGRLLYTLIDNVVGYLGK

SEQ ID NO: 21 - protein, 526 aa, Cutaneotrichosporon oleaginosum
MATLYPSIQSLQKFPYPGDGVVSSTLTDQHDTEGLIADVLDEQPPAHVPRLGLQNATTTLDSVNHLKFIQGAMMSLPSGFVGLDASRPWLVFWTVHSLDLLGVLLPQNIRDRAVSTILHFLHPTGGFCGGAANTHMPHLLPTYASVVSLAIVGNAGKGGGWERLVDARQDIYNFFMRCKRPDGGFVVGDNCEVDVRGTYCLLVVATLLDIITPELLHNVDKAIAAGQTFEGGFACSSFTFKDGNRVAMSEAHGGYTSCSVFSHFLLSSVQPPRRLESLPESFPVPIDVDSVVRWSAMMQGEAADGGGFRGRSNKLVDGCYSWWVGGTFPVLEELRRREAEVKTSPNGPTATKIVAVDDDGEDEWADEASMHALFNRGMCDSEVRLMAVALQEYTLLVAQSVTRGGLRDKPGKGPDLYHTCNNLSGLSVAQHRLTHTPEEVQKQREAFKADRGLPAVKPTTPGGGWKSEEERQAARREVWANVRAWVEDESDTLVVGGQMSQVNTTVPPFNMLEVRLQPFIDYFYCQ

SEQ ID NO: 22 - protein, 345 aa, Sapingoeca rosetta
MGYDGLVKLDPEQHLPYVTGGLGTLPSGFETLDASRPWLVYWSLNALVILGGTISPELKRRVINTLRMCQAETGGFGGGVGQVAHAAPTYAAVNALAIIGTEEAWSIINREKLASWLSSLIEDDGSMHMHDDGEIDVRAVYCGASAARLCGLDVDTIFAKCPQWVARCQTYEGGFAAIPGLEAHGGYTFCGFAAMSILCSTHLIDIPRLTEWLANRQMPMSGGFQGRPNKLVDGCYSFWVGGCFPILADLLEAQGLPGDVVNAEALIDYVVCVCQCPSGFRDKPGKRQDYYHTSYCLSGLASMKRFAPNHPILSQLNATHPIHNVPPANAERMIQAMSSQTTTRH